(19) United States
(12) Patent Application Publication
(10) Pub. No.: US 2025/0281570 A1
(43) Pub. Date: Sep. 11, 2025

(54) Treatment of Myasthenia Gravis with Zilucoplan

(71) Applicants: UCB BIOPHARMA SRL, Brussels (BE); RA PHARMACEUTICALS, INC., Cambridge, MA (US)

(72) Inventors: Babak Boroojerdi, Brussels (BE); Petra Duda, Charlestown, MA (US); Melissa Kaye Brock, Brussels (BE)

(21) Appl. No.: 18/862,147

(22) PCT Filed: May 5, 2023
(86) PCT No.: PCT/US2023/021199

§ 371 Date: Oct. 31, 2024

Related U.S. Application Data
(60) Provisional application No. 63/338,824, filed on May 5, 2022.

(51) Int. Cl. A61K 38/12; A61K 9/00; A61K 45/06; A61P 21/04

(52) U.S. Cl. CPC A61K 38/12; A61K 9/0019; A61K 45/06; A61P 21/04

(57) ABSTRACT

The present disclosure provides methods of using zilucoplan as a therapy for AChR positive gMG patients who have gMG refractory to conventional immunosuppressive therapy and/or intravenous (IV) immunoglobulin and plasma exchange (PLEX) therapy.

### CROSS-REFERENCE TO RELATED APPLICATIONS

This application claims the benefit of U.S. Provisional Application No. 63/338,824, filed May 5, 2022, which is hereby incorporated in its entirety by reference.

### BACKGROUND

Myasthenia gravis (MG) is a rare chronic complement-mediated autoimmune disease characterized by the production of autoantibodies targeting proteins that are critical for the normal transmission of electrical signals from nerves to muscles. The most common target of autoantibodies in MG is the nicotinic acetylcholine receptor (AChR), located at the post junctional membrane of the neuromuscular junction (NMJ), the point at which a motor neuron transmits chemical signals to a skeletal muscle fiber. The prevalence of MG in the United States is estimated at approximately 60,000 cases. In approximately 15% of patients with MG, symptoms are confined to the ocular muscles. The remaining patients have MG that affects multiple muscle groups throughout the body, which is typically referred to as generalized MG (gMG). Patients with gMG present with muscle weakness that characteristically becomes more severe with repeated use and recovers with rest. Muscle weakness can be localized to specific muscles, but often progresses to more diffuse muscle weakness. Generalized myasthenia gravis symptoms can become life-threatening when muscle weakness involves the diaphragm and intercostal muscles in the chest wall that are responsible for breathing. The most dangerous complication of gMG, known as myasthenic crisis, requires hospitalization, intubation, and mechanical ventilation. Approximately 15% to 20% of patients with gMG will experience a myasthenic crisis within 2 years of diagnosis.

Despite treatment, many patients are unable to achieve control of their gMG symptoms and the burden of gMG can be attributed to both the disease and the conventional treatments. The symptom burden associated with gMG means that many patients require support from professional caregivers and family and friends with activities of daily living. Current standard of care (SOC) therapy for gMG includes multiple categories of therapeutics. However, each therapeutic agent or category of therapeutics has their challenges and limitations.

The approval of eculizumab does not address the needs of all gMG patients. Multiple national MG patient registries have demonstrated that many patients still experience significant disease burden (e.g., QMG≥12 or MG-ADL≥6), regardless of current treatment.

There is an ongoing need for novel therapies for patients who continue to have a unmet medical needs and disease burden despite standard of care (SOC) therapies. The goal of gMG treatment is to achieve remission and/or improve symptoms of excessive/chronic muscle fatigue, reducing the risk of life threatening acute respiratory failure, lowering the risk of mortality and thereby allowing a patient to have an improved quality of life.

### SUMMARY

Zilucoplan is a synthetic, macrocyclic peptide that binds complement component 5 (C5) with sub-nanomolar affinity and allosterically inhibits its cleavage into C5a and C5b upon activation of the classical, alternative, or lectin pathways. Like the C5 inhibitory monoclonal antibody eculizumab, zilucoplan blocks the proteolytic cleavage of C5 into C5a and C5b. Unlike eculizumab, zilucoplan can also bind to C5b and block C6 binding which prevents the subsequent assembly of the MAC.

Zilucoplan has been clinically assessed for the treatment of conditions in which C5 activation has been demonstrated to play a role, including generalized myasthenia gravis (gMG). In a Phase 2, 44 patient clinical study of gMG (NCT03315130), both high and low dose zilucoplan treatments were shown to be effective with a favorable safety profile, and higher doses yielding more robust clinical improvement.

In the RAISE Phase 3 clinical study (NCT04115293) described herein, the efficacy and safety of zilucoplan is studied in patients with gMG, including both patients having gMG refractory to SOC therapies, and patients having non-refractory gMG. The present disclosure presents results and analysis of the RAISE study.

Analysis of the RAISE clinical study results indicates that treatment of patients having refractory gMG with zilucoplan at week 12 achieve an improved response as compared to the response achieved by eculizumab at week 26 of the corresponding REGAIN Phase 3 study (Howard et al., 2017).

Accordingly, the present disclosure provides methods of using zilucoplan as a therapy for acetylcholine receptor antibody positive (AChR+) gMG patients who have refractory gMG, i.e., gMG refractory to conventional immunosuppressive therapy and/or intravenous (IV) immunoglobulin and plasma exchange (PLEX) therapy (e.g., as described herein).

### BRIEF DESCRIPTION OF THE FIGURES

The foregoing and other objects, features and advantages will be apparent from the following description of particular embodiments of the invention, as illustrated in the accompanying drawings. The drawings are not necessarily to scale; emphasis instead being placed upon illustrating the principles of various disclosed embodiments.

FIG. 1 is a schematic showing a myasthenia gravis treatment RAISE Phase 3 clinical study design for myasthenia gravis treatment using zilucoplan.

FIG. 2 shows the change from baseline (95% CI) over time of the MG-Activities of Daily Living (MG-ADL) score in the modified intent to treat (mITT) population of the RAISE clinical study. CFB=change from baseline; CI=confidence interval; LS=least squares; LSM=least squares mean.

FIG. 3 shows the change from baseline (95% CI) over time of the quantitative myasthenia gravis (QMG) score in the modified intent to treat (mITT) population of the RAISE clinical study.

FIG. 4 shows the change from baseline (95% CI) over time of the myasthenia gravis composite (MGC) score in the modified intent to treat (mITT) population of the RAISE clinical study.

FIG. 5 shows the change from baseline (95% CI) over time of the myasthenia gravis quality of life 15 revised (MG QOL15r) score in the modified intent to treat (mITT) population of the RAISE clinical study.

FIG. 6 shows the time to first receipt of rescue therapy in the modified intent to treat (mITT) population of the RAISE clinical study.

FIGS. 7A-7C illustrates responder rates for MG-ADL (FIG. 7A), QMG score (FIG. 7B), and minimal symptom expression (MSE, MG-ADL score of 0 or 1) (FIG. 7C) without rescue therapy at week 12 for placebo versus zilucoplan. Significantly more participants treated with ZLP achieved a ≥3 point reduction in MG-ADL score.

FIG. 8 shows a responder analysis for changes in MG-ADL score at week 12 in the modified intent to treat (mITT) population of the RAISE clinical study.

FIG. 9 shows a responder analysis for changes in QMG score at week 12 in the modified intent to treat (mITT) population of the RAISE clinical study.

FIGS. 10A-10D illustrates baseline characteristics (continuous) for MG-ADL responders and non-responders at Week 12. FIG. 10A, age as baseline. FIG. 10B, age at diagnosis. FIG. 10C, disease duration at baseline. FIG. 10D, MG-ADL score at baseline. CI, confidence interval.

FIGS. 11A-11C illustrates baseline characteristics (categorical) for MG-ADL responders and non-responders at Week 12. FIG. 11A, gender (male). FIG. 11B, refractory status (yes). FIG. 11C, at least 2 prior MG therapies, excluding acetylcholinesterase inhibitor (AChEI). CI, confidence interval.

### DETAILED DESCRIPTION

Complement activity protects the body from foreign pathogens but can lead to self-cell destruction with elevated activity or poor regulation. Generalized myasthenia gravis (gMG) is a neurological disorder characterized by autoantibody-mediated nervous system destruction. The present disclosure relates to particular methods of treating gMG by administering the complement C5 inhibitor zilucoplan. These and other embodiments of the disclosure are described in detail below.

### I. Zilucoplan Compounds and Compositions

Zilucoplan and compositions including zilucoplan which function to modulate complement activity (e.g., inhibit complement C5 activation) are useful in the methods of the present disclosure.

Zilucoplan is a polypeptide. The core amino acid sequence of zilucoplan ([cyclo(1,6)]Ac-K-V-E-R-F-D-(N-Me)D-Tbg-Y-azaTrp-E-Y-P-Chg-K; SEQ ID NO: 1) includes 15 amino acids (all L-amino acids), including 4 non-natural amino acids [N-methyl-aspartic acid or “(N-Me)D”, tert-butylglycine or “Tbg”, 7-azatryptophan or “azaTrp”, and cyclohexylglycine or “Chg” ]; a lactam bridge between K1 and D6 of the polypeptide sequence; and a C-terminal lysine residue with a modified side chain, forming a N-ε-(PEG24-γ-glutamic acid-N-α-hexadecanoyl)lysine residue (also referred to herein as “B28”). The C-terminal lysine side chain modification includes a polyethyleneglycol (PEG) spacer (PEG24), with the PEG24 being attached to an L-γ glutamic acid residue that is derivatized with a palmitoyl group.

“B28” refers to N-ε-(PEG24-γ-glutamic acid-N-α-hexadecanoyl)lysine.

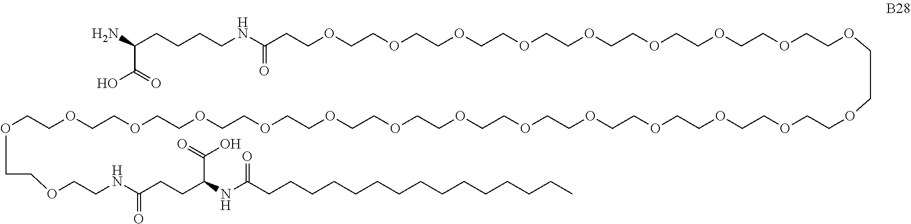 [SMILES: CCCCCCCCCCCCCCCC(=O)N[C@@H](CCC(=O)NCCOCCOCCOCCOCCOCCOCCOCCOCCOCCOCCOCCOCCOCCOCCOCCOCCOCCOCCOCCOCCOCCOCCOCCOCCC(=O)NCCCC[C@H](N)C(=O)O)C(=O)O]

The free acid form of zilucoplan has a molecular formula of C₁₇₂H₂₇₈N₂₄O₅₅, a molecular weight of 3562.23 Daltons (Da), and an exact mass of 3559.97 amu (see CAS Number 1841136-73-9). The tetra sodium form of zilucoplan has a molecular formula of C₁₇₂H₂₇₈N₂₄O₅₅Na₄, and can be referred to by the following chemical name: Acetyl-[L-Lysyl¹-L-Valyl²-L-Glutamyl³-L-Arginyl⁴-L Phenylalanyl⁵-L Aspartyl⁶]-N-Methyl-L-Aspartyl⁷-L-tert-Leucyl⁸-L-Tyrosyl⁹-L-7-Azatryptophyl¹⁰-L-Glutamyl¹¹-L-Tyrosyl¹²-L-Prolyl¹³-L-Cyclohexylglycyl¹⁴-[L-Lysyl¹⁵, Nε-Palmitoyl-γ-L-Glutamyl-(1-Amino-3,6,9,12,15,18,21,24,27,30, 33,36,39,42,45,48,51,54,57,60,63,66,69,72-tetracosaoxapentaheptacontan-75-oyl)], cyclic (lactam 1-6), tetra sodium.

The chemical structure of sodium salt form of zilucoplan is shown in structure I.

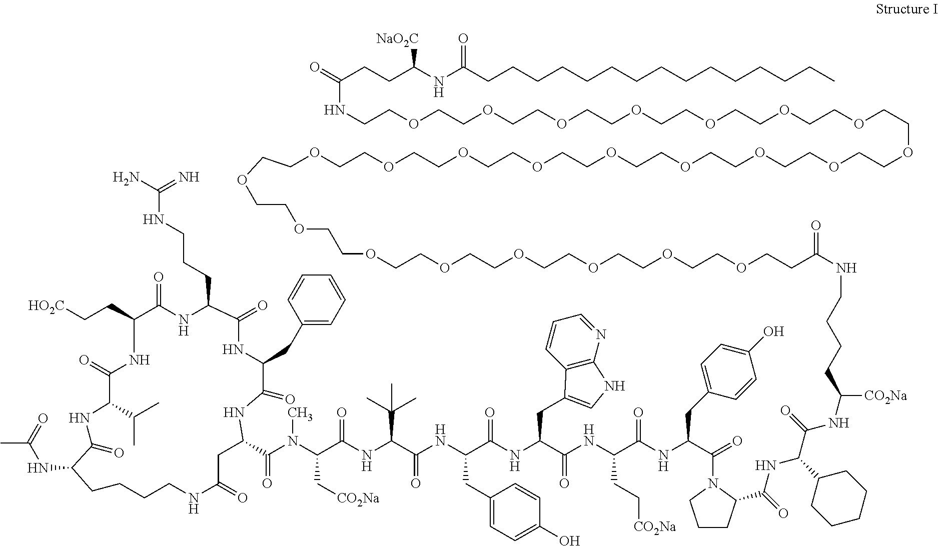

The four sodium ions in the structure are shown associated with designated carboxylates, but they may be associated with any of the acidic groups in the molecule. In some embodiments, the zilucoplan drug substance is typically provided as the sodium salt form and is lyophilized. In some embodiments, the zilucoplan drug substance is formulated as a sterile, preservative-free solution to be administered via subcutaneous (SC) injection. In some embodiments, the zilucoplan solution is provided in a single-use injectable, passive needle safety device. The free base form of zilucoplan or any pharmaceutically acceptable salt of zilucoplan are encompassed by the term “zilucoplan”.

### Isotopic Variations

Compounds of the present disclosure may include one or more atoms that are isotopes. The term “isotope” refers to a chemical element that has one or more additional neutrons. In some embodiments, compounds of the present disclosure may be deuterated. The term “deuterated” refers to a substance that has had one or more hydrogen atoms replaced by deuterium isotopes. Deuterium isotopes are isotopes of hydrogen. The nucleus of hydrogen contains one proton while deuterium nuclei contain both a proton and a neutron. Compounds and compositions of the present disclosure may be deuterated in order to change a physical property, such as stability, or to allow for use in diagnostic and experimental applications.

### II. Methods of Treating Myasthenia Gravis

The present disclosure provides methods related to using zilucoplan compounds and compositions for therapeutic treatment of generalized myasthenia gravis.

Generalized myasthenia gravis (gMG) is a rare complement-mediated autoimmune disease characterized by the production of autoantibodies targeting proteins that are critical for the normal transmission of chemical or neurotransmitter signals from nerves to muscles, e.g., acetylcholine receptor (AChR) proteins. The presence of AChR autoantibodies in patient samples can be used as an indicator of disease. While about 15% of patients have symptoms that are confined to ocular muscles, the majority of patients experience generalized myasthenia gravis.

In some embodiments, the patient treated according to the methods of this disclosure is an adult patient who is anti-acetylcholine receptor (AchR) antibody positive.

Generalized myasthenia gravis (gMG) refers to MG that affects multiple muscle groups throughout the body. Although the prognosis of gMG is generally benign, 10% to 15% of patients have refractory gMG. The clinical classification from the Myasthenia Gravis Foundation of America (MGFA) for the purpose of grouping patients according to their disease severity is as follows:
Class I: Any ocular muscle weakness; may have weakness of eye closure. All other muscle strength is normal.
Class II: Mild weakness affecting muscles other than ocular muscles; may also have ocular muscle weakness of any severity.
IIa. Predominantly affecting limb, axial muscles, or both. May also have lesser involvement of oropharyngeal muscles.
IIb. Predominantly affecting oropharyngeal, respiratory muscles, or both. May also have lesser or equal involvement of limb, axial muscles, or both.
Class III: Moderate weakness affecting muscles other than ocular muscles; may also have ocular muscle weakness of any severity.
IIIa. Predominantly affecting limb, axial muscles, or both. May also have lesser involvement of oropharyngeal muscles.
IIIb. Predominantly affecting oropharyngeal, respiratory muscles, or both. May also have lesser or equal involvement of limb, axial muscles, or both.
Class IV: Severe weakness affecting muscles other than ocular muscles; may also have ocular muscle weakness of any severity.
IVa. Predominantly affecting limb, axial muscles, or both. May also have lesser involvement of oropharyngeal muscles.
IVb. Predominantly affecting oropharyngeal, respiratory muscles, or both. May also have lesser or equal involvement of limb, axial muscles, or both.
Class V: Defined as intubation, with or without mechanical ventilation, except when employed during routine postoperative management. The use of a feeding tube without intubation places the patient in class IVb.

Refractory gMG refers to gMG where disease control either cannot be achieved with conventional therapies, and/or results in severe side effects of immunosuppressive therapy. This severe form of gMG affects approximately 9,000 individuals in the United States. Various criteria are used to characterize treatment-refractory gMG disease (see e.g., Mantegazza and Antozzi, “When myasthenia gravis is deemed refractory: clinical signposts and treatment strategies”, Ther. Adv. Neurol. Disord. Jan. 18, 2018, 1-11, DOI: 10.1177/1756285617749134).

As per the inclusion criteria for the eculizumab Phase 3 study (REGAIN study), the treatment refractory gMG status of patients treated according to the methods of this disclosure can be defined as:
a) Failed treatment over 1 year or more with two (2) or more immunosuppressive therapies (ISTs) either in combination or as monotherapy (i.e., continued to have impairment of activities of daily living [persistent weakness, experienced crisis, or unable to tolerate IST] despite ISTs); and/or
b) Failed at least 1 IST and required chronic plasmapheresis, plasma exchange (PLEX), or IVIg to control symptoms (i.e., a study participant who required PLEX and/or IVIg on a regular basis for the management of muscle weakness at least every 3 months over the previous 12 months).

In some embodiments of the method, the patient is refractory to treatment for 1 year or more with immunosuppressant therapy (IST) and requires chronic plasma exchange or chronic IVIG to maintain clinical stability.

Non-refractory gMG refers to gMG where disease control can be achieved with conventional therapies, i.e., the patient does not meet either of the inclusion criteria a) or b) above. In some embodiments, a patient having non-refractory gMG is a patient who has received standard of care (SOC) gMG therapy (e.g., immunosuppressive therapy) for less than 1 year. In some embodiments, the patient responds to standard of care gMG therapy. The SOC gMG therapy can be an immunosuppressant therapy (e.g, steroidal and/or non-steroidal) that provided maintenance, control and/or reduction of symptoms for the patient.

Patients with gMG present with muscle weakness that characteristically becomes more severe with repeated use and recovers with rest. Muscle weakness can be localized to specific muscles, such as those responsible for eye movements, but often progresses to more diffuse muscle weakness. gMG may even become life-threatening when muscle weakness involves the diaphragm and the other chest wall muscles responsible for breathing. This is the most feared complication of gMG, known as myasthenic crisis or MG crisis, and requires hospitalization, intubation, and mechanical ventilation. Approximately 15% to 20% of patients with gMG experience a myasthenic crisis within two years of diagnosis.

The most common target of autoantibodies in gMG is the acetylcholine receptor, or AChR, located at the neuromuscular junction, the point at which a motor neuron transmits signals to a skeletal muscle fiber. Current therapies for gMG focus on either augmenting the AChR signal or nonspecifically suppressing the autoimmune response. First-line therapy for symptomatic gMG is treatment with acetylcholinesterase inhibitors such as pyridostigmine. Although sometimes adequate for control of mild ocular symptoms, pyridostigmine monotherapy is usually insufficient for the treatment of generalized weakness, and dosing of this therapy may be limited by cholinergic side effects. Therefore, in patients who remain symptomatic despite pyridostigmine therapy, corticosteroids with or without systemic immunosuppressives are indicated (Sanders D B, et al. 2016. Neurology. 87(4):419-25). Immunosuppressives used in gMG include azathioprine, cyclosporine, mycophenolate mofetil, methotrexate, tacrolimus, cyclophosphamide, and rituximab. These agents are associated with well-documented long-term toxicities. Surgical removal of the thymus may be recommended in patients with nonthymomatous gMG and moderate to severe symptoms in an effort to reduce the production of AChR autoantibodies. Intravenous immunoglobulin (IVIg) and plasma exchange (PLEX) are usually restricted to short-term use in patients with myasthenic crisis or life-threatening signs such as respiratory insufficiency or dysphagia.

In a Phase 2, randomized, double-blind, placebo-controlled trial, eculizumab was tested in 14 AChR autoantibody-positive patients with refractory gMG, who had a quantitative myasthenia gravis (QMG) score ≥12 and previously failed treatment with at least 2 immunosuppressant therapies (ISTs) (Howard, J F. 2013. Myasthenia Gravis Foundation of America. Clinical Overview of MG, the content of which is herein incorporated by reference in its entirety). Patients were randomized in a 1:1 ratio to receive either eculizumab or placebo. Patients on eculizumab received 600 mg per week for 4 weeks, followed by 900 mg every other week by IV infusion, for a total of 16 weeks of treatment. After a 5-week washout period, patients were crossed over to the opposite arm of the study. Patients who received placebo for the first 16 weeks of the study were treated with eculizumab and vice versa. The primary endpoints were safety and efficacy, as measured by the percentage of patients who achieved a ≥3-point reduction in QMG score. The impact of C5 inhibition by eculizumab in QMG score occurred rapidly (within 1 week of initiating treatment) and favored eculizumab compared with placebo across all study visits (p=0.0144). Following the initial 16-week treatment period, 6 out of 7 patients on eculizumab achieved a ≥3-point improvement in QMG score, compared with 4 out of 7 patients in the placebo arm. Of those patients who responded to eculizumab, 4 achieved an 8-point reduction in QMG score compared with only 1 in the placebo arm.

The QMG is a standardized and validated quantitative strength scoring system developed for gMG and is used in clinical trials as an endpoint of interest. The scoring system assesses 13 items relating to ocular, bulbar, and limb function (Barnet, C. et al. 2015. J Neuromuscul. Dis. 2:301-11). Each item is scored from 0-3. Maximum total score is 39. Higher scores are representative of more severe impairment. Recent data suggest that improvements in the QMG score of 2 to 3 points may be considered clinically meaningful, depending upon disease severity [Barohn R J et al. 1998. Ann NY Acad Sci. 841:769-772; Katzberg H D et al. 2014. Muscle Nerve. 49(5):661-665].

A Phase 3 trial (NCT01997229) was also completed that enrolled 125 AChR autoantibody-positive patients with a Myasthenia Gravis-Activities of Daily Living (MG-ADL) score ≥6, who had previously failed 2 ISTs or had failed 1 IST and required chronic plasma exchange or IV immunoglobulin therapy. The MG-ADL is a brief 8-item survey designed to evaluate gMG symptom severity. Each item is scored from 0-3. Maximum total score is 24. Higher scores are associated with more severe symptoms of gMG. The MG-ADL has been shown to correlate with other validated gMG outcome measures (e.g., MG-QOL15r), and a 2-point improvement in MG-ADL score is considered clinically meaningful [Wolfe G I et al. 1999. Neurology. 52(7):1487-9; Muppidi S et al. 2011. Muscle Nerve. 44(5):727-31]. The MG-QOL15r is a 15-item survey that was designed to assess quality of life in patients with gMG based on patient reporting. Each item is scored from 0-2. Maximum total score is 30. Higher scores indicate more severe impact of the disease on aspects of the patient's life [Burns, T M et al. 2010. Muscle Nerve. 41(2):219-26; Burns™ et al. 2016. Muscle Nerve. 54(6):1015-22].

Patients were randomized 1:1 to receive either placebo or eculizumab for a 26-week treatment period, followed by an extension study. Patients receiving eculizumab were treated with 900 mg per week for 4 weeks followed by 1200 mg every other week by IV infusion. Eculizumab treatment was not associated with a statistically significant benefit relative to placebo in the primary endpoint of change from baseline in MG-ADL (p=0.0698) in this study. However, statistically significant results were observed in 18 of 22 prespecified analyses, including the secondary endpoint of change from baseline in QMG score (p=0.0129).

The present disclosure provides methods of treating gMG by inhibiting C5 activity in a subject. “C5-dependent complement activity” or “C5 activity” refers to activation of the complement cascade through cleavage of C5, the assembly of downstream cleavage products of C5, or any other process or event attendant to, or resulting from, the cleavage of C5. In some cases, the percentage of C5 activity inhibited in a subject may be at least 10%, at least 20%, at least 30%, at least 40%, at least 50%, at least 60%, at least 70%, at least 80%, at least, 85%, at least 90%, at least 95%, at least 96%, at least 97%, at least 98%, at least 99%, at least 99.5%, or at least 99.9%.

C5 inhibitor zilucoplan may be used to treat gMG, wherein few or no adverse effects result from treatment. In some cases, no adverse cardiovascular, respiratory, and/or central nervous system (CNS) effects occur. In some cases, no changes in heart rate and/or arterial blood pressure occur. In some cases, no changes to respiratory rate, tidal volume, and/or minute volume occur.

A treatment or preventive effect is evident when there is a significant improvement, often statistically significant, in one or more parameters of disease status, or by a failure to worsen or to develop symptoms where they would otherwise be anticipated. As an example, a favorable change of at least 10% in a measurable parameter of disease, and at least 20%, 30%, 40%, 50% or more can be indicative of effective treatment. Efficacy for a given compound or composition can also be judged using an experimental animal model for the given disease as known in the art. When using an experimental animal model, efficacy of treatment is evidenced when a statistically significant modulation in a marker or symptom is observed.

In some embodiments, the present disclosure provides methods of inhibiting C5 activity in a tissue by contacting the tissue with a tissue-penetrating C5 inhibitor, e.g., zilucoplan. The term “tissue-penetrating” refers to a property characterized by tissue permeability. Agents with enhanced tissue-penetration may demonstrate better distribution in tissues when compared to agents with less or no tissue-penetration. Tissue penetration may be assessed by ability to cross basement membranes. The term “basement membrane” refers to an extracellular matrix (ECM) protein layer separating endothelial cells from underlying tissues. Tissue penetration assessments may be done in vivo or in vitro and may include the use of basement membrane models. Such models may include measuring compound diffusion across artificial basement membranes. Such models may include the use of upper and lower reservoirs separated by an artificial basement membrane. Artificial basement membranes may include any of the ECM gel membranes described in Arends, F. et al. 2016. IntechOpen, DOI: 10.5772/62519, the contents of which are herein incorporated by reference in their entirety. ECM gel membranes may be prepared to include matrix components mimicking those found in the basal lamina of neuromuscular junctions. In some models, compounds being tested are introduced to upper reservoirs and compound diffusion is detected in lower reservoirs.

Tissue penetration assessment may include visual assessments e.g., through use of fluorescent labels to visualize analyte movement across basement membranes. Some assessments may include biochemical analysis of samples obtained from the penetrated side of a basement membrane.

In some embodiments, compound permeability may be determined using quantitative whole body analysis (QWBA). QWBA is a form of analysis that uses radiography to assess distribution of radiolabeled analytes. In some embodiments, radiolabeled compounds are administered to subjects and tissue distribution of the compounds is analyzed over time.

Contacting tissues with the tissue-penetrating C5 inhibitor may include administering zilucoplan to tissues as part of a formulation. Such formulations may be administered by subcutaneous injection. Tissue-penetrating C5 inhibitors may be able to penetrate basement membranes. Basement membrane permeability of polypeptide tissue-penetrating C5 inhibitors may be greater than basement membrane permeability of larger proteins, such as antibodies. Such advantages may be due to restrictively large size of proteins and antibodies. Zilucoplan basement membrane permeability may be from about 3-fold to about 5-fold greater than basement membrane permeability of eculizumab, offering advantages over eculizumab for inhibiting C5 activity in tissues and treating related complement-related indications. In some embodiments, zilucoplan permeability enhances distribution in one or more of lung, heart, muscle, small intestine, large intestine, spleen, liver, bone, stomach, lymph node, fat, brain, pancreas, testes, and thymus, in comparison to eculizumab.

Polypeptide-based C5 inhibitors (e.g., zilucoplan) may be used to treat complement-related indications (e.g., myasthenia gravis) benefiting from rapid and/or enhanced inhibitor tissue distribution. The tissue may include muscle and/or neuromuscular junction (NMJ). Polypeptide inhibitors (e.g., zilucoplan) may provide superior penetration into muscle and/or NMJ compared to antibodies based on smaller size and/or favorable charge profile. Such penetration may lead to faster relief from overactive complement. Further, polypeptide inhibitor (e.g., zilucoplan) penetration may stabilize and/or improve NMJ membrane potential by preventing MAC pore formation. Accordingly, safety factor at the NMJ may be improved. The term “safety factor” refers to excess transmitter levels released after nerve impulse that ensure neuromuscular transmission effectiveness under physiological stress. The excess is the amount beyond that required to trigger muscle fiber action potential and contributes to membrane potential restoration.

### Combination Therapy

Zilucoplan and additional therapeutic agent(s) and/or therapies can be administered in combination. Such combinations may be in the same composition, or the additional therapeutic agents or therapies can be administered as part of a separate composition or by another method described herein.

During administration of zilucoplan according to methods of this disclosure, subjects may receive standard of care (SOC) therapy for gMG. Standard of care therapies for gMG may include, but are not limited to, plasma exchange (PLEX), intravenous immunoglobin (IVIg) treatment, biologics (e.g., rituximab or eculizumab or ravulizumab), pyridostigmine treatment, corticosteroid treatment, and/or immunosuppressive (e.g., non-steroidal immunosuppressive) drug treatment.

In some embodiments of the methods of this disclosure, subjects are co-administered zilucoplan and one or more steroid and/or or non-steroidal immunosuppressant (e.g., as described herein). In some embodiments, zilucoplan is co-adminstered to a subject identified as having side-effects and/or at risk of adverse events caused by immunosuppressive therapy. In some embodiments, zilucoplan is co-administered with a reduced dosage of a steroid and/or or non-steroidal immunosuppressive therapy to treat the gMG with an acceptable side-effect and adverse-event profile. By reduced dosage is meant a dosage reaction as compared to a conventional dosage under standard of care (SOC) immunosuppressive therapy for gMG. In some embodiments, the steroid is a corticosteroid. In some embodiments, the immunosuppressant is selected from azathioprine, cyclosporine, mycophenolate mofetil, methotrexate, tacrolimus, cyclophosphamide, and rituximab.

In some embodiments, subjects receive cholinesterase inhibitor treatment over the course of zilucoplan treatment.

Cyclosporine A is a known immunosuppressive agent, inhibitor of organic anion transporting polypeptide (OATP) 1B1 and OATP1B3, and is a potential comedication in PNH and other complement-related indications. In some embodiments, cyclosporine A and zilucoplan may be administered in combination to subjects with myasthenia gravis. Cyclosporine A and zilucoplan may be administered in overlapping dosage regimens. Other immunosuppressive agents that may be administered in combination with or in overlapping dosage regiments with zilucoplan may include, but are not limited to, azathioprine, cyclosporine, mycophenolate mofetil, methotrexate, tacrolimus, cyclophosphamide, and rituximab.

In some embodiments, the present disclosure provides methods of treating gMG in subjects by administering zilucoplan in combination with neonatal Fc receptor (FcRN) inhibitor treatments. FcRN inhibitor treatments may be used to treat autoimmune diseases that include autoantibody-mediated tissue destruction. FcRN inhibitor treatments may include intravenous immunoglobulin (IVIG) treatment, which reduces the half-life of IgG antibodies by overwhelming the Fc recycling mechanism with large doses of immunoglobulin. Some FcRN inhibitor treatments may include administration of DX-2504 or functionally equivalent variants thereof, e.g., DX-2507, which includes modifications to reduce aggregation and improve manufacturability (described in Nixon, A. E. et al. 2015. Front Immunol. 6:176). DX-2504 is an inhibitor of FcRN recycling. By inhibiting FcRN, DX-2504 inhibits Fc-mediated recycling, thereby reducing the half-life of IgG antibodies. Administration of DX-2504 may also be used in models of IVIG treatment. In some embodiments, zilucoplan may be administered to treat complement-related indications (e.g., myasthenia gravis) in overlapping dosage regimens with FcRN inhibitor treatments. The FcRN inhibitor treatments may include DX-2504 (or DX-2507) administration and/or IVIG treatment.

### Screening and Diagnosis

Patients treated with zilucoplan according to the methods of this disclosure may be screened prior to zilucoplan administration. The terms “patient”, “subject” and “individual” are used interchangeably herein. The term “screen” refers to a review or evaluation carried out for the purpose of selection or filtration. Patients may be screened to select individuals in need of treatment. In some embodiments, subjects are screened to select individuals most likely to respond favorably to treatment.

Screening may include selecting subjects previously diagnosed with gMG. The gMG diagnosis may be made according to Myasthenia Gravis Foundation of America (MGFA) criteria; Class II-IVa (see Howard, J. F., 2009. Myasthenia Gravis A Manual for the Health Care Provider, Myasthenia Gravis Foundation of America, Inc.).

In some embodiments of the methods of this disclosure, screening includes assessing whether a human patient is positive for auto-antibodies binding to nicotinic acetylcholine receptor (anti-AChR).

In some embodiments of the methods of this disclosure, screening includes assessing whether a human patient has refractory gMG.

In some embodiments of the methods of this disclosure, screening includes assessing whether a human patient has non-refractory gMG.

In some embodiments, screening may be carried out to identify subjects with a stage of gMG that occurs prior to reaching a critical or crisis stage. Such screening may be carried out to identify subjects prior to developing gMG or early in the disease process that may benefit from proactive or preventative treatment.

In some embodiments, screening is carried out to exclude individuals with greater risks associated with treatment.

In some embodiments, the patient is identified as having refractory gMG.

Screening may include assessment of one or more quantitative scoring systems of disease severity for gMG. Subjects receiving conventional or SOC gMG therapies prior to or during screening may be maintained on such therapies during the screening process or may be required to withhold one or more treatments before or during the screening process. In some embodiments, a period of time between prior gMG therapy and a screening assessment is required. The period of time may be required to obtain reliable results from a particular screening assessment.

Screening may include selecting subjects based on age. In some embodiments, screening may be carried out to select subjects with ages between 18 and 85 years old. In some embodiments, screening may be carried out to select subjects 18 to 74 years old.

Screening may include assessment of biomarker levels. In some embodiments, biomarkers include acetylcholinesterase receptor (AChR) autoantibody levels. AChR autoantibodies may lead to disease by binding AChR and stimulating complement activation. Accordingly, AChR autoantibody levels may be a good indicator of complement-mediated disease. In some embodiments, biomarkers include autoantibodies to muscle-specific tyrosine kinase (MuSK). Subjects with anti-MuSK antibodies are part of a distinct MG subset associated with less predictable treatment outcomes (Lavrnic, D. et al. 2005. J Neurol Neurosurg Psychiatry. 76:1099-102). Screening may include excluding subjects with anti-MuSK antibodies from treatment and/or evaluations.

Screening may include review of subject prior and current treatments. In some embodiments, subjects are screened based on recent changes in treatments. In some embodiments, subjects are screened to confirm no change in corticosteroid dose or immunosuppressive therapy prior to screening. The screening may exclude subjects from treatment where subject corticosteroid treatment dose or immunosuppressive therapy regimen changes within the 30 days prior to screening.

Subjects may be screened for pregnancy status. In some embodiments, pregnant subjects may be excluded from treatment. Pregnancy status screening may be carried out by serum pregnancy test. In some embodiments, pregnancy screening may include urine pregnancy testing.

### Zilucoplan Treatment

Zilucoplan inhibits C5a formation in a dose-dependent manner upon activation of the classical pathway and inhibits C5b formation (as measured by C5b-9 or MAC deposition on a complement activating surface) upon activation of the classical and alternative complement pathways.

Methods of the present disclosure include methods of treating gMG by zilucoplan administration to a subject. Zilucoplan administration may be subcutaneous (SC) administration. Zilucoplan may be administered at a dose of from about 0.01 mg/kg (mg zilucoplan/kg subject body weight) to about 1.0 mg/kg, from about 0.02 mg/kg to about 2.0 mg/kg, or from about 0.05 mg/kg to about 3.0 mg/kg.

Methods of the present disclosure may include administering zilucoplan at a daily dose of from about 0.1 mg/kg to about 0.3 mg/kg. In some embodiments, zilucoplan is administered at a daily dose of 0.3 mg/kg.

Zilucoplan administration may be by self-administration. Zilucoplan administration may include the use of prefilled syringes. Self-administration may include the use of self-administration devices. Self-administration devices may include or be incorporated with prefilled syringes.

Zilucoplan may be provided in solution. Zilucoplan solutions may include aqueous solutions. Zilucoplan solutions may include phosphate-buffered saline (PBS). Zilucoplan solutions may be preservative-free. Zilucoplan may be present in solution at a concentration of from about 1 mg/mL to about 400 mg/mL, such as from about 4 mg/ml to about 200 mg/ml, from about 1 mg/mL to about 5 mg/mL, from about 2 mg/mL to about 10 mg/mL, or from about 10 mg/mL to about 50 mg/mL zilucoplan. In some embodiments, the solution includes about 40 mg/ml zilucoplan.

In some embodiments, self-administration devices include zilucoplan solutions. In some embodiments, the self-administration device has a maximum fill volume of at least 1 ml. Self-administration devices may include zilucoplan solution volumes of from about 0.010 mL to about 0.500 mL, from about 0.050 mL to about 0.600 mL, from about 0.100 mL to about 0.700 mL, from about 0.150 mL to about 0.810 mL, from about 0.200 mL to about 0.900 mL, or from about 0.250 mL to about 1.00 mL. In some embodiments, the self-administration devices includes a zilucoplan solution of from about 0.15 mL to about 0.81 mL.

Zilucoplan treatment may include administration of one or more doses. In some embodiments, treatment is in doses that occur hourly, twice daily, daily, bi-daily, weekly, bi-weekly, monthly, or combinations thereof. Zilucoplan treatment may include daily administration. Subject zilucoplan plasma levels may reach maximum concentration (Cₘₐₓ) on a first day of treatment. Serum hemolysis may be inhibited by zilucoplan treatment. In some embodiments, at least 90% hemolysis inhibition is achieved in subject serum with zilucoplan treatment.

Zilucoplan treatment for gMG may be carried out with a variety of subjects from different demographic backgrounds and stages of disease. Refractory subjects may include those who have been resistant or unresponsive to prior therapy with eculizumab.

In some embodiments, subjects with a stage of gMG that occurs prior to reaching a critical or crisis stage are treated with zilucoplan. Such treatment may be carried out to treat subjects prior to developing gMG or early in the disease process to provide benefits of proactive or preventative treatment.

In some embodiments, the present invention provides zilucoplan for use in a method of treating gMG comprising administering 0.1 to 0.3 mg/kg zilucoplan subcutaneously or intravenously to a subject. In some embodiments, the present invention provides zilucoplan for use in a method of treating gMG comprising administering 0.1 mg/kg or 0.3 mg/kg zilucoplan subcutaneously or intravenously to the subject. In some embodiments, the present invention provides zilucoplan for use in a method of treating refractory gMG comprising administering 0.3 mg/kg zilucoplan subcutaneously to the subject.

### Evaluation

Subjects receiving zilucoplan treatment for gMG may be evaluated for efficacy during or after treatment. The term “treated subject” refers to an individual that has received at least one treatment. Zilucoplan treated subject evaluation may include evaluation of one or more metrics of efficacy. In some embodiments, evaluations may require subject treatments to be withheld for a period prior to evaluation. Some evaluations may require subjects to maintain consistent treatments before, during, and/or after evaluations. Withheld or maintained treatments may be zilucoplan treatments. In some embodiments, withheld or maintained treatments include other treatments for gMG or for non-MG conditions.

Evaluations may be carried out to assess primary efficacy endpoints. The term “primary endpoint” refers to a result that answers the most important inquiry addressed by a particular study. The term “secondary endpoint,” refers to a result that answers other relevant inquiries subordinate to a main inquiry. A primary efficacy endpoint is a result that addresses whether or not a treatment is effective, while a secondary efficacy endpoint addresses one or more peripheral inquiries (e.g., quality of life impact, side effect severity, etc.).

Evaluations may be carried out to assess subject gMG characteristics. The term “gMG characteristic” refers to a physical or mental trait or set of traits associated with the presence of or severity of gMG in a subject. gMG characteristics may include scores obtained using different disease evaluation methods. gMG characteristics may include, but are not limited to, Myasthenia Gravis Activities of Daily Living (MG-ADL) score, Myasthenia Gravis score (QMG), in Myasthenia Gravis Composite (MGC) score, or in quality of life as measured by Myasthenia Gravis Quality of Life revised (MG-QOL-15r) score. In some embodiments, subjects may be monitored for gMG characteristics over time. Such monitoring may be carried out over the course of gMG disease. Monitoring may be carried out over the course of disease treatment. In some embodiments, subject evaluation or monitoring is carried out to assess changes in gMG characteristics during or after subject treatment with zilucoplan. Such changes can be assessed via comparison to a baseline characteristic or score determined before treatment with zilucoplan begins (e.g., as described herein).

In some embodiments, zilucoplan treated subjects are evaluated or monitored for MG-ADL score. Change in MG-ADL score from baseline may be a primary efficacy endpoint. The MG-ADL is a brief 8-item survey designed to evaluate gMG symptom severity. Each item is scored from 0-3. Maximum total score is 24. Higher scores are associated with more severe symptoms of gMG. The MG-ADL has been shown to correlate with other validated gMG outcome measures (e.g., MG-QOL15r), and a 2-point improvement in MG-ADL score is considered clinically meaningful [Wolfe G I et al. 1999. Neurology. 52(7):1487-9; Muppidi S et al. 2011. Muscle Nerve. 44(5):727-31, the contents of which are herein incorporated by reference in their entirety].

In some embodiments, zilucoplan treated subjects are evaluated or monitored for QMG score. The QMG is a standardized and validated quantitative strength scoring system that was developed specifically for gMG and has been used previously in clinical trials. The scoring system assesses 13 items relating to ocular, bulbar, and limb function (Barnet, C. et al. 2015. J Neuromuscul Dis. 2:301-11). Each item is scored from 0-3. Maximum total score is 39. Higher scores are representative of more severe impairment. Recent data suggest that improvements in the QMG score of 2 to 3 points may be considered clinically meaningful, depending upon disease severity [Barohn R J et al. 1998. Ann N Y Acad Sci. 841:769-772; Katzberg H D et al. 2014. Muscle Nerve. 49(5):661-665, the contents of which are herein incorporated by reference in their entirety]. Subjects being assessed for QMG score may be pulled from gMG therapies for at least 10 hours prior to QMG score assessment. The gMG therapies may include acetylcholinesterase inhibitor therapy (e.g., pyridostigmine treatment) for at least 10 hours prior to QMG score assessment.

Change in QMG score from baseline may be a primary or secondary efficacy endpoint. Treated subject QMG score may be reduced. The QMG score may be reduced by at least 3 points. The QMG score may be reduced after completing 12 weeks of zilucoplan treatment. The QMG score may be reduced at or before 12 weeks of zilucoplan treatment. Treated subject QMG score may be monitored over the course of zilucoplan treatment.

In some embodiments, zilucoplan treated subject evaluations may include testing and/or monitoring for one or more of MG-ADL score, QMG score, MG-QOL15r score, and MG Composite score. Such scores may be evaluated as primary or secondary efficacy endpoints. The MG-QOL15r is a 15-item survey that was designed to assess quality of life in patients with gMG based on patient reporting. Each item is scored from 0-2. Maximum total score is 30. Higher scores indicate more severe impact of the disease on aspects of patient life [Burns, T M et al. 2010. Muscle Nerve. 41(2):219-26; Burns™ et al. 2016. Muscle Nerve. 54(6):1015-22, the contents of which are herein incorporated by reference in their entirety]. The MG Composite is a 10-item scale that has been used to measure the clinical status of patients with gMG, both in the practice setting and in clinical trials, in order to evaluate treatment response (Burns, T. M. et al., 2008. Muscle Nerve. 38:1553-62). 10 items are assessed related to ocular, bulbar, respiratory, neck, and limb function. Items weighted, with scores ranging from 0-9. Maximum total score is 50. Higher scores in the MG Composite indicate more severe impairment due to the disease. A 3-point change in this instrument is considered clinically meaningful [Burns, T. M. et al. 2010. Neurology. 74(18): 1434-40; Sadjadi, D B et al. 2012. Neurology. 2016; 87(4):419-425, the contents of which are herein incorporated by reference in their entirety].

Testing or monitoring for MG-ADL, QMG, MG-QOL15r, and/or MG Composite scores may be used to identify changes from baseline score. The term “baseline score” refers to a score obtained before initial treatment. Baseline scores may be scores obtained between a switch from one treatment to another. The switch may be from a placebo to an active pharmaceutical compound. In some embodiments, zilucoplan treatment may be evaluated for reduction in MG-ADL score of at least 2 points. The reduction may occur at or before 12 weeks of zilucoplan treatment. In some embodiments, zilucoplan treatment may be evaluated for reduction in MG Composite score of at least 3 points. The reduction may occur at or before 12 weeks of zilucoplan treatment.

In some embodiments, zilucoplan treatment leads to reduced subject symptom expression. The reduced subject symptom expression may exceed reduced subject symptom expression associated with eculizumab administration.

### Evaluation Methods

In some embodiments, the present disclosure provides methods of evaluating treatments for gMG. Such methods may include screening evaluation candidates for at least one evaluation participation criteria. The term “evaluation candidate” refers to any individual being considered for participation in an evaluation (e.g., a clinical study). “Evaluation participation criteria” refers to a metric or factor used to select individuals to include in an evaluation. Evaluation candidates selected for participation in an evaluation are referred to herein as “evaluation participants.” In some embodiments, methods of evaluating treatments for gMG may include screening an evaluation candidate for at least one evaluation participation criteria; selecting an evaluation participant; administering the treatment for gMG to the evaluation participant; and assessing at least one efficacy endpoint.

In some embodiments, evaluation participation criteria include gMG diagnosis. Diagnosis of gMG may be made according to MGFA criteria. In some embodiments, evaluation participation criteria include QMG score. Evaluation participant selections may require evaluation candidate QMG scores of ≥12. Some evaluation candidates may have received at least one alternative gMG treatment (i.e., alternative to the treatment for gMG being tested, such as standard of care treatments) prior to screening. In some embodiments, such candidates may be assessed for QMG score at least 10 hours after most recent alternative gMG treatment. Alternative gMG treatments may include standard of care gMG treatments, including, but not limited to, cholinesterase inhibitor treatment, acetylcholinesterase inhibitor treatment, pyridostigmine treatment, corticosteroid treatment, and immunosuppressive drug treatment. Evaluation participant selection may require a score of ≥2 for ≥4 QMG test items.

In some embodiments, evaluation participation criteria include evaluation candidate age. In some embodiments, evaluation candidates must be between 18 and 85 years old. In some embodiments, evaluation candidates must be 18 to 74 years old.

Evaluation participation criteria may include candidate biomarker levels. In some embodiments, biomarkers include acetylcholinesterase receptor (AChR) autoantibody levels. AChR autoantibodies may lead to disease by binding AChR and stimulating complement activation. Accordingly, AChR autoantibody levels may be a good indicator of susceptibility to complement-mediated disease.

Evaluation participation criteria may include candidate prior and current alternative gMG treatment status. In some embodiments, evaluation participants are selected based consistency of current or former alternative gMG treatments. In some embodiments, candidates with no recent change in corticosteroid dose or immunosuppressive therapy are selected. Candidates with corticosteroid treatment dose or immunosuppressive therapy regimen changes within the past 30 days may be excluded from evaluation participation.

Evaluation participation criteria may include pregnancy status. In some embodiments, pregnant subjects may be excluded from evaluation participation. Pregnancy status screening may be carried out by serum pregnancy test. In some embodiments, pregnancy screening may include urine pregnancy testing.

Methods of evaluating treatments for gMG may include administering treatments for gMG to evaluation participants over an evaluation period. As used herein, the term “evaluation period” refers to a time frame over which a particular study takes place. Treatments may be administered over evaluation periods of from about one day to about 24 weeks. Some evaluation periods are about 12 weeks or longer. Evaluation participants may continue to receive standard of care gMG therapies over evaluation periods. Such therapies may include, but are not limited to, cholinesterase inhibitor treatment, acetylcholinesterase inhibitor treatment, pyridostigmine treatment, corticosteroid treatment, and/or immunosuppressive drug treatment.

Efficacy endpoints may include certain scores or changes in scores associated with assessments for individuals with gMG. Such assessments may include, but are not limited to, QMG score, MG-ADL score, MG-QOL15r score, and MG Composite score. In some embodiments, efficacy endpoints include QMG score reduction. Efficacy endpoints may include at least 3 point reductions in QMG score. For evaluation participants receiving alternative gMG treatments (e.g., acetylcholinesterase inhibitor treatment) during the evaluation period, one or more of those treatments may be withheld for at least 10 hours prior to QMG score assessment. In some embodiments, efficacy endpoints include reduction in one or more of MG-ADL score, MG-QOL15r score, and MG Composite score in relation to baseline score. Efficacy endpoints may include 2-point reduction in MG-ADL score over baseline score. The reduction in MG-ADL score may occur at or before 12 weeks of treatment for gMG.

In some embodiments, assessing efficacy endpoints includes a set of assessments. The set of assessments may be carried out in a particular order. In some embodiments, the set of assessments are carried out in the order of: (1) assessing evaluation participant MG-QOL15r score; (2) assessing evaluation participant MG-ADL score; (3) assessing evaluation participant QMG score; and (4) assessing evaluation participant MG Composite score.

Assessments for efficacy endpoints may be carried out on one or more occasions after administering treatments for gMG. Such assessments may be carried out at specific times and/or dates or may be carried out on a recurring basis (e.g., hourly, daily, weekly, monthly, or combinations thereof). In some embodiments, assessments are carried out 1 week, 2 weeks, 4 weeks, 8 weeks, and/or 12 weeks after starting administration of treatments for gMG.

In some embodiments of the methods of this disclosure, the patient is identified as having refractory gMG and experiences a clinically meaningful improvement (reduction) in quantitative Myasthenia Gravis (QMG) score, in Myasthenia Gravis Composite (MGC) score, or in quality of life as measured by Myasthenia Gravis Quality of Life revised (MG-QOL-15r) score, after 12 weeks of treatment. In some embodiments, the clinically meaningful improvement is experienced at or before 12 weeks of zilucoplan treatment. In some embodiments, the clinically meaningful improvement is experienced at or before 8 weeks of zilucoplan treatment.

In some embodiments, the patient identified as having refractory gMG experiences a reduction in Myasthenia Gravis Activities of Daily Living (MG-ADL) score of at least 3 points from baseline after 12 weeks of zilucoplan treatment (e.g., at least 4 points, at least 5 points, at least 6 points, at least 8, at least 10, or at least 12 points reduction from baseline). In some embodiments, the reduction from baseline of at least 3 points in MG-ADL score is achieved or experienced at or before 12 weeks of zilucoplan treatment. In some embodiments, the reduction from baseline of at least 3 points in MG-ADL score is achieved or experienced after 8 weeks of zilucoplan treatment.

In some embodiments, the patient identified as having refractory gMG experiences a reduction in QMG score of at least 3 points from baseline after 12 weeks of treatment (e.g., at least 4 points, at least 5 points, at least 6 points, at least 8, at least 10, or at least 12 points reduction from baseline). In some embodiments, the reduction from baseline of at least 3 points in QMG score is achieved or experienced at or before 12 weeks of zilucoplan treatment. In some embodiments, the reduction from baseline of at least 3 points in QMG score is achieved or experienced after 8 weeks of zilucoplan treatment.

In some embodiments, the patient identified as having refractory gMG experiences a reduction in MGC score of at least 3 points from baseline after 12 weeks of treatment (e.g., at least 4 points, at least 5 points, at least 6 points, at least 8, at least 10, or at least 12 points reduction from baseline). In some embodiments, the reduction from baseline of at least 3 points in MGC score is achieved or experienced at or before 12 weeks of zilucoplan treatment.

In some embodiments, the patient identified as having refractory gMG experiences a reduction in MG-QOL-15r score of at least 2 points from baseline after 12 weeks of treatment (e.g., at least 3 points, at least 4 points, at least 5 points, at least 6 points, at least 8, at least 10, or at least 12 points reduction from baseline). In some embodiments, the reduction from baseline of at least 2 points in MG-QOL-15r score is achieved or experienced at or before 12 weeks of zilucoplan treatment.

### Pharmaceutical Compositions

In some embodiments, C5 inhibitor zilucoplan compositions are pharmaceutical compositions that include at least one pharmaceutically acceptable excipient. In some embodiments, the pharmaceutically acceptable excipient includes at least one of a salt and a buffering agent. The salt may be sodium chloride. The buffering agent may be sodium phosphate. Sodium chloride may be present at a concentration of from about 0.1 mM to about 1000 mM. In some cases, sodium chloride may be present at a concentration of from about 25 mM to about 100 mM. Sodium phosphate may be present at a concentration of from about 0.1 mM to about 1000 mM. In some cases, sodium phosphate is present at a concentration of from about 10 mM to about 100 mM.

In some embodiments, zilucoplan compositions include a solution including from about 0.01 mg/mL to about 4000 mg/mL of zilucoplan. In some cases, zilucoplan is present at a concentration of from about 1 mg/mL to about 400 mg/mL, such as from about 4 mg/ml to about 200 mg/mL. In some embodiments, zilucoplan compositions includes a solution of about 40 mg/mL zilucoplan.

Compositions of the present disclosure may include zilucoplan at a concentration of approximately, about or exactly any of the following values: 0.001 mg/mL, 0.2 mg/mL, 0.01 mg/mL, 2 mg/mL, 0.1 mg/mL, 10 mg/mL, 0.5 mg/mL, 5 mg/mL, 1 mg/mL, 20 mg/mL, 15 mg/mL, 40 mg/mL, 25 mg/mL, 75 mg/mL, 50 mg/mL, 200 mg/mL, 100 mg/mL, or 400 mg/mL. In some cases, compositions include zilucoplan at a concentration of at least 40 mg/mL.

In some embodiments, compounds or compositions, e.g., pharmaceutical compositions, of the present disclosure are formulated in aqueous solutions. In some cases, aqueous solutions further include one or more salt and/or one or more buffering agent. Salts may include sodium chloride which may be included at concentrations of from about 0.05 mM to about 50 mM, from about 1 mM to about 100 mM, from about 20 mM to about 200 mM, or from about 50 mM to about 500 mM. Further solutions may include at least 500 mM sodium chloride. In some cases, aqueous solutions include sodium phosphate. Sodium phosphate may be included in aqueous solutions at a concentration of from about 0.005 mM to about 5 mM, from about 0.01 mM to about 10 mM, from about 0.1 mM to about 50 mM, from about 1 mM to about 100 mM, from about 5 mM to about 150 mM, or from about 10 mM to about 250 mM. In some cases, at least 250 mM sodium phosphate concentrations are used.

Aqueous zilucoplan formulations may have pH levels of from about 2.0 to about 3.0, from about 2.5 to about 3.5, from about 3.0 to about 4.0, from about 3.5 to about 4.5, from about 4.0 to about 5.0, from about 4.5 to about 5.5, from about 5.0 to about 6.0, from about 5.5 to about 6.5, from about 6.0 to about 7.0, from about 6.5 to about 7.5, from about 7.0 to about 8.0, from about 7.5 to about 8.5, from about 8.0 to about 9.0, from about 8.5 to about 9.5, or from about 9.0 to about 10.0.

### Dosage and Administration

For treatment of human subjects, C5 inhibitors (e.g., zilucoplan) may be formulated as pharmaceutical compositions. Depending on the subject to be treated, the mode of administration, and the type of treatment desired (e.g., prevention, prophylaxis, or therapy) C5 inhibitors (e.g., zilucoplan) may be formulated in ways consonant with these parameters. A summary of such techniques is found in Remington: The Science and Practice of Pharmacy, 21st Edition, Lippincott Williams & Wilkins, (2005); and Encyclopedia of Pharmaceutical Technology, eds. J. Swarbrick and J. C. Boylan, 1988-1999, Marcel Dekker, New York, each of which is incorporated herein by reference.

C5 inhibitors (e.g., zilucoplan) may be provided in a therapeutically effective amount. In some cases, a therapeutically effective amount of a C5 inhibitor (e.g., zilucoplan) may be achieved by administration of a dose of from about 0.1 mg to about 1 mg, from about 0.5 mg to about 5 mg, from about 1 mg to about 20 mg, from about 5 mg to about 50 mg, from about 10 mg to about 100 mg, from about 20 mg to about 200 mg, or at least 200 mg of one or more C5 inhibitors (e.g., zilucoplan).

In some embodiments, subjects may be administered a therapeutic amount of a C5 inhibitor (e.g., zilucoplan) based on the weight of such subjects. In some cases, zilucoplan is administered at a dose of from about 0.1 mg/kg to about 3 mg/kg. Such ranges may include ranges suitable for administration to human subjects. Dosage levels may be highly dependent on the nature of the condition; drug efficacy; the condition of the patient; the judgment of the practitioner; and the frequency and mode of administration.

In some cases, C5 inhibitors (e.g., zilucoplan) is provided at concentrations adjusted to achieve a desired level of the C5 inhibitor in a sample, biological system, or subject (e.g., plasma level in a subject). In other embodiments, C5 inhibitors are administered at a dose sufficient to achieve a maximum serum concentration (Cₘₐₓ) of at least 0.1 μg/mL, at least 0.5 μg/mL, at least 1 μg/mL, at least 5 μg/mL, at least 10 μg/mL, at least 50 μg/mL, at least 100 μg/mL, or at least 1000 μg/mL.

In some embodiments, C5 inhibitors (e.g., zilucoplan and/or active metabolites or variants thereof) are administered daily at a dose sufficient to deliver from about 0.1 mg/day to about 60 mg/day per kg weight of a subject. In some cases, the Cₘₐₓ achieved with each dose is from about 0.1 μg/mL to about 1000 μg/mL. In such cases, the area under the curve (AUC) between doses may be from about 200 μg*hr/mL to about 10,000 μg*hr/mL.

According to some methods of the present disclosure, zilucoplan is provided at a concentration needed to achieve a desired effect. In some cases, compounds and compositions of the disclosure are provided at an amount necessary to reduce a given reaction or process by half. The concentration needed to achieve such a reduction is referred to herein as the half maximal inhibitory concentration, or “IC₅₀.” Alternatively, compounds and compositions of the disclosure may be provided at an amount necessary to increase a given reaction, activity or process by half. The concentration needed for such an increase is referred to herein as the half maximal effective concentration or “EC₅₀.”

C5 inhibitors (e.g., zilucoplan) may be present in amounts totaling 0.1-95% by weight of the total weight of the composition. In some cases, C5 inhibitors are provided by intravenous (IV) administration. In some cases, C5 inhibitors are provided by subcutaneous (SC) administration.

SC administration of C5 inhibitors (e.g., zilucoplan) may, in some cases, provide advantages over IV administration. SC administration may include self-administration by using an administration device, such as a self-administration device. As used herein, the term “self-administration” refers to any form of therapeutic delivery that is carried out wholly or in part by the recipient of a therapeutic treatment. Self-administration devices may include self-injection devices. Self-administration treatment may be advantageous in that patients can provide treatment to themselves in their own home, avoiding the need to travel to a provider or medical facility. Further, SC treatment may allow patients to avoid long-term complications associated with IV administration, such as infections, loss of venous access, local thrombosis, and hematomas. In some embodiments, self-administration using a self-injection device may increase patient compliance, patient satisfaction, quality of life, reduce treatment costs and/or drug requirements.

In some cases, daily SC administration provides steady-state C5 inhibitor concentrations that are reached within 1-3 doses, 2-3 doses, 3-5 doses, or 5-10 doses. In some cases, daily SC doses of from about 0.1 mg/kg to about 0.3 mg/kg may achieve sustained C5 inhibitor levels greater than or equal to 2.5 μg/mL and/or inhibition of complement activity of greater than 90%.

C5 inhibitors (e.g., zilucoplan) may exhibit slow absorption kinetics (time to maximum observed concentration of greater than 4-8 hours) and high bioavailability (from about 75% to about 100%) after SC administration.

In some embodiments, dosage and/or administration are altered to modulate the half-life (t_{1/2}) of C5 inhibitor (e.g., zilucoplan) levels in a subject or in subject fluids (e.g., plasma).

In some embodiments, C5 inhibitors (e.g., zilucoplan) may exhibit long terminal t_{1/2}. Extended terminal t_{1/2} may be due to extensive target binding and/or additional plasma protein binding. In some cases, C5 inhibitors exhibit t_{1/2} values greater than 24 hours in both plasma and whole blood. In some cases, C5 inhibitors do not lose functional activity after incubation in human whole blood at 37° C. for 16 hours.

In some embodiments, dosage and/or administration are altered to modulate the steady state volume of distribution of C5 inhibitors. In some cases, the dosage and/or administration of C5 inhibitors is adjusted to ensure that the steady state volume of distribution is equal to at least 50% of total blood volume. In some embodiments, C5 inhibitor distribution may be restricted to the plasma compartment.

Time periods for which maximum concentration of C5 inhibitors in subjects (e.g., in subject serum) are maintained (Tₘₐₓ values) may be adjusted by altering dosage and/or administration (e.g., subcutaneous administration).

In some embodiments, C5 inhibitors (e.g., zilucoplan) may be administered without off-target effects. In some cases, C5 inhibitors do not inhibit hERG (human ether-a-go-go related gene), even with concentrations less than or equal to 300 μM. SC injection of C5 inhibitors with dose levels up to 10 mg/kg may be well tolerated and not result in any adverse effects of the cardiovascular system (e.g., elevated risk of prolonged ventricular repolarization) and/or respiratory system.

In some embodiments, C5 inhibitors of the present disclosure allow for a rapid washout period in cases of infection where prolonged inhibition of the complement system prove detrimental.

C5 inhibitor administration according to the present disclosure may be modified to reduce potential clinical risks to subjects. Infection with Neisseria meningitidis is a known risk of C5 inhibitors, including eculizumab. In some cases, risk of infection with Neisseria meningitides is minimized by instituting one or more prophylactic steps. Such steps may include the exclusion of subjects who may already be colonized by these bacteria. In some cases, prophylactic steps may include coadministration with one or more antibiotics. In some cases, ciprofloxacin may be co-administered. In some cases, ciprofloxacin may be co-administered orally at a dose of from about 100 mg to about 1000 mg (e.g., 500 mg).

In some embodiments, C5 inhibitors (e.g., zilucoplan) are administered at a frequency of every hour, every 2 hrs, every 4 hrs, every 6 hrs, every 12 hrs, every 18 hrs, every 24 hrs, every 36 hrs, every 72 hrs, every 84 hrs, every 96 hrs, every 5 days, every 7 days, every 10 days, every 14 days, every week, every two weeks, every 3 weeks, every 4 weeks, every month, every 2 months, every 3 months, every 4 months, every 5 months, every 6 months, every year, or at least every year. In some cases, C5 inhibitors are administered once daily or administered as two, three, or more sub-doses at appropriate intervals throughout the day.

In some embodiments, C5 inhibitors are administered in multiple daily doses. In some cases, C5 inhibitors are administered daily for 7 days. In some cases, C5 inhibitors are administered daily for 7 to 100 days. In some cases, C5 inhibitors are administered daily for at least 100 days. In some cases, C5 inhibitors are administered daily for an indefinite period.

Methods of the present disclosure may include administering a C5 inhibitor (e.g., zilucoplan) at a daily dose of from about 0.1 mg/kg to about 0.3 mg/kg. In some embodiments, a C5 inhibitor (e.g., zilucoplan) is administered at a daily dose of 0.3 mg/kg.

C5 inhibitors delivered intravenously may be delivered by infusion over a period of time, such as over a 5 minute, 10 minute, 15 minute, 20 minute, or 25 minute period. The administration may be repeated, for example, on a regular basis, such as hourly, daily, weekly, biweekly (i.e., every two weeks), for one month, two months, three months, four months, or more than four months. After an initial treatment regimen, treatments may be administered on a less frequent basis. For example, after biweekly administration for three months, administration may be repeated once per month, for six months or a year or longer.

Before administration of a full dose of C5 inhibitor and/or C5 inhibitor composition, patients can be administered a smaller dose, such as 5% of a full dose, and monitored for adverse effects, such as an allergic reaction or infusion reaction, or for elevated lipid levels or blood pressure. In another example, patients can be monitored for unwanted immunostimulatory effects, such as increased cytokine (e.g., TNF-alpha, IL-1, IL-6, or IL-10) levels.

Genetic predisposition plays a role in the development of some diseases or disorders. Therefore, patients in need of C5 inhibitors may be identified by family history analysis, or, for example, screening for one or more genetic markers or variants. Healthcare providers (e.g., doctors or nurses) or family members may analyze family history information before prescribing or administering therapeutic compositions of the present disclosure.

### III. Kits and Devices

In some embodiments, the present disclosure provides kits and devices. Such kits and devices may include any of the compounds or compositions described herein. In a non-limiting example, zilucoplan may be included.

Devices of the present disclosure may include administration devices. As used herein, the term “administration device” refers to any tool for providing a substance to a recipient. Administration devices may include self-administration devices. As used herein, the term “self-administration device” refers to any tool used for providing a substance to a recipient, wherein use of the tool is carried out wholly or in part by the recipient. Self-administration devices may include self-injection devices. “Self-injection devices” are self-administration devices that enable individuals to subcutaneously administer substances to their own body. Self-injection devices may include prefilled syringes. As used herein, the term “prefilled syringe” refers to a syringe that has been loaded with a substance or cargo prior to access or use by an operator of the syringe. For example, prefilled syringes (also referred to herein as “pre-loaded syringes”) may be filled with a therapeutic composition prior to packaging in a kit; prior to syringe shipment to a distributor, administrator, or operator; or prior to access by a subject using the syringe for self-administration. Due to cyclic peptide stability, cyclic peptide inhibitors (e.g., zilucoplan) are especially well suited for manufacture, storage, and distribution in pre-loaded syringes. Further, pre-loaded syringes are especially well suited for self-administration (i.e., administration by a subject, without the aid of a medical professional). Self-administration represents a convenient way for subjects to obtain treatments without relying on medical professionals who may be located at a distance or are otherwise difficult to access. This makes self-administration options well suited for treatments requiring frequent injections (e.g., daily injections).

Prefilled syringes may be of any material (e.g., glass, plastic, or metal). In some embodiments, prefilled syringes are glass syringes. Prefilled syringes may include maximum fill volumes (meaning the largest amount of liquid that can be contained) of at least 0.1 ml, at least 0.2 ml, at least 0.3 ml, at least 0.4 ml, at least 0.5 ml, at least 0.75 ml, at least 1.0 ml, at least 1.5 ml, at least 2.0 ml, at least 5.0 ml, at least 10 ml, or more than 10 ml. Syringes may include needles. The needles may be of any gauge. In some embodiments, syringes include 29-gauge needles. The needles may be assembled with syringes or attached prior to syringe use. Self-injection devices may include BD ULTRASAFE PLUS™ self-administration devices (BD, Franklin Lakes, NJ).

Administration devices may include self-injection devices that include a syringe and needle and a predetermined volume of a zilucoplan composition. The zilucoplan composition may be a pharmaceutical composition. The composition may include a zilucoplan concentration of from about 1 mg/mL to about 200 mg/mL. In some embodiments, the zilucoplan concentration is about 40 mg/mL. Predetermined volumes may be predetermined based on subject body weight. In some embodiments, predetermined zilucoplan composition volumes are modified to facilitate zilucoplan administration to a subject at a dose of from about 0.1 mg/kg to about 0.6 mg/kg. Volumes may be modified to facilitate 0.3 mg/kg zilucoplan dosing. The self-injection device may include a BD ULTRASAFE PLUS™ self-administration device. In some embodiments, administration devices are prepared for storage at specific temperatures or temperature ranges. Some administration devices may be prepared for storage at room temperature. Some administration devices may be prepared for storage between from about 2° C. to about 8° C.

Pre-filled syringes may include ULTRASAFE PLUS™ passive needle guards (Becton Dickenson, Franklin Lakes, NJ). Other pre-filled syringes may include injection pens. Injection pens may be multi-dose pens. Some pre-filled syringes may include a needle. In some embodiments, the needle gauge is from about 20 to about 34. The needle gauge may be from about 29 to about 31.

In some embodiments, kits of the present disclosure include kits carrying out methods of treating MG described herein. Such kits may include one or more administration devices described herein and instructions for kit usage.

Kit components may be packaged in liquid (e.g., aqueous or organic) media or in dry (e.g., lyophilized) form. Kits may include containers that may include, but are not limited to vials, test tubes, flasks, bottles, syringes, or bags. Kit containers may be used to aliquot, store, preserve, insulate, and/or protect kit components. Kit components may be packaged together or separately. Some kits may include containers of sterile, pharmaceutically acceptable buffer and/or other diluent (e.g., phosphate buffered saline). In some embodiments, kits include containers of kit components in dry form with separate containers of solution for dissolving dried components. In some embodiments, kits include a syringe for administering one or more kit components.

When polypeptides are provided as a dried powder it is contemplated that between 10 micrograms and 1000 milligrams of polypeptide, or at least or at most those amounts are provided in kits.

Containers may include at least one vial, test tube, flask, bottle, syringe and/or other receptacle, into which polypeptide formulations may be placed, preferably, suitably allocated. Kits may also include containers for sterile, pharmaceutically acceptable buffer and/or other diluent.

Kits may include instructions for employing kit components as well the use of any other reagent not included in the kit. Instructions may include variations that can be implemented.

Kits may include one or more items for addressing syringe wounds. Such items may include, but are not limited to, alcohol wipes and wound dressings (e.g., cotton balls, mesh pads, bandages, tape, gauze, etc.). Kits may further include disposal containers for disposal of used kit components. Disposal containers may be designed for disposal of sharp objects, such as needles and syringes. Some kits may include instructions for sharp object disposal.

In some embodiments, kits of the present disclosure include zilucoplan in powdered form or in solution (e.g., as pharmaceutical compositions). Solutions may be aqueous solutions. Solutions may include PBS. Zilucoplan solutions may include from about 4 mg/ml to about 200 mg/ml zilucoplan. In some embodiments, zilucoplan solutions include about 40 mg/ml zilucoplan. Zilucoplan solutions may include preservatives. In some embodiments, zilucoplan solutions are preservative-free.

In some embodiments, kits are prepared for storage at specific temperatures or temperature ranges. Some kits may be prepared for storage at room temperature. Some kits may be prepared for storage between from about 2° C. to about 8° C.

### IV. Definitions

Bioavailability: As used herein, the term “bioavailability” refers to the systemic availability of a given amount of a compound (e.g., C5 inhibitor) administered to a subject. Bioavailability can be assessed by measuring the area under the curve (AUC) or the maximum serum or plasma concentration (Cₘₐₓ) of the unchanged form of a compound following administration of the compound to a subject. AUC is a determination of the area under the curve when plotting the serum or plasma concentration of a compound along the ordinate (Y-axis) against time along the abscissa (X-axis). Generally, the AUC for a particular compound can be calculated using methods known to those of ordinary skill in the art and/or as described in G. S. Banker, Modern Pharmaceutics, Drugs and the Pharmaceutical Sciences, v. 72, Marcel Dekker, New York, Inc., 1996, the contents of which are herein incorporated by reference in their entirety.

Biological system: As used herein, the term “biological system” refers to a cell, a group of cells, a tissue, an organ, a group of organs, an organelle, a biological fluid, a biological signaling pathway (e.g., a receptor-activated signaling pathway, a charge-activated signaling pathway, a metabolic pathway, a cellular signaling pathway, etc.), a group of proteins, a group of nucleic acids, or a group of molecules (including, but not limited to biomolecules) that carry out at least one biological function or biological task within cellular membranes, cellular compartments, cells, cell cultures, tissues, organs, organ systems, organisms, multicellular organisms, biological fluids, or any biological entities. In some embodiments, biological systems are cell signaling pathways that include intracellular and/or extracellular signaling biomolecules. In some embodiments, biological systems include proteolytic cascades (e.g., the complement cascade).

Buffering agent: The term “buffering agent” refers to a compound used in a solution for the purposes of resisting changes in pH. Such compounds may include, but are not limited to acetic acid, adipic acid, sodium acetate, benzoic acid, citric acid, sodium benzoate, maleic acid, sodium phosphate, tartaric acid, lactic acid, potassium metaphosphate, glycine, sodium bicarbonate, potassium phosphate, sodium citrate, and sodium tartrate.

Clearance rate: The term “clearance rate” refers to the velocity at which a particular compound is cleared from a biological system or fluid.

Complement activity includes the activation of the complement cascade, the formation of cleavage products from a complement component such as C3 or C5, the assembly of downstream complexes following a cleavage event, or any process or event attendant to, or resulting from, the cleavage of a complement component, e.g., C3 or C5. Complement inhibitors may include C5 inhibitors that block complement activation at the level of complement component C5. C5 inhibitors may bind C5 and prevent its cleavage, by C5 convertase, into the cleavage products C5a and C5b.

Complement component C5 or C5 is defined as a complex which is cleaved by C5 convertase into at least the cleavage products, C5a and C5b.

C5 inhibitors include any compound or composition that inhibits the processing or cleavage of the pre-cleaved complement component C5 complex or the cleavage products of the complement component C5. It is understood that inhibition of C5 cleavage prevents the assembly and activity of the cytolytic membrane attack complex (MAC) on glycosylphosphatidylinositol (GPI) adherent protein-deficient erythrocytes. In some cases, C5 inhibitors presented herein may also bind C5b, preventing C6 binding and subsequent assembly of the C5b-9 MAC.

Compound: The term “compound,” refers to a distinct chemical entity. In some embodiments, a particular compound may exist in one or more isomeric or isotopic forms (including, but not limited to stereoisomers, geometric isomers and isotopes). In some embodiments, a compound is provided or utilized in only a single such form. In some embodiments, a compound is provided or utilized as a mixture of two or more such forms (including, but not limited to a racemic mixture of stereoisomers). Those of skill in the art will appreciate that some compounds exist in different forms, show different properties and/or activities (including, but not limited to biological activities). In such cases it is within the ordinary skill of those in the art to select or avoid particular forms of a compound for use in accordance with the present disclosure. For example, compounds that contain asymmetrically substituted carbon atoms can be isolated in optically active or racemic forms.

Cyclic or Cyclized: As used herein, the term “cyclic” refers to the presence of a continuous loop. Cyclic molecules need not be circular, only joined to form an unbroken chain of subunits. Cyclic polypeptides may include a “cyclic loop,” formed when two amino acids are connected by a bridging moiety. The cyclic loop comprises the amino acids along the polypeptide present between the bridged amino acids. Cyclic loops may include 2, 3, 4, 5, 6, 7, 8, 9, 10 or more amino acids.

Downstream event: As used herein, the term “downstream” or “downstream event,” refers to any event occurring after and/or as a result of another event. In some cases, downstream events are events occurring after and as a result of C5 cleavage and/or complement activation. Such events may include, but are not limited to, generation of C5 cleavage products, activation of MAC, hemolysis, and hemolysis-related disease (e.g., PNH).

Equilibrium dissociation constant: As used herein, the term “equilibrium dissociation constant” or “K_{D}” refers to a value representing the tendency of two or more agents (e.g., two proteins) to reversibly separate. In some cases, K_{D} indicates a concentration of a primary agent at which half of the total levels of a secondary agent are associated with the primary agent.

Half-life: As used herein, the term “half-life” or “t_{1/2}” refers to the time it takes for a given process or compound concentration to reach half of a final value. The “terminal half-life” or “terminal t_{1/2}” refers to the time needed for the plasma concentration of a factor to be reduced by half after the concentration of the factor has reached a pseudo-equilibrium.

Identity: As used herein, the term “identity,” when referring to polypeptides or nucleic acids, refers to a comparative relationship between sequences. The term is used to describe the degree of sequence relatedness between polymeric sequences and may include the percentage of matching monomeric components with gap alignments (if any) addressed by a particular mathematical model or computer program (i.e., “algorithms”). Identity of related polypeptides can be readily calculated by known methods. Such methods include, but are not limited to, those described previously by others (Lesk, A. M., ed., Computational Molecular Biology, Oxford University Press, New York, 1988; Smith, D. W., ed., Biocomputing: Informatics and Genome Projects, Academic Press, New York, 1993; Griffin, A. M. et al., ed., Computer Analysis of Sequence Data, Part 1, Humana Press, New Jersey, 1994; von Heinje, G., Sequence Analysis in Molecular Biology, Academic Press, 1987; Gribskov, M. et al., ed., Sequence Analysis Primer, M. Stockton Press, New York, 1991; and Carillo et al., Applied Math, SIAM J, 1988, 48, 1073).

Inhibitor: As used herein, the term “inhibitor” refers to any agent that blocks or causes a reduction in the occurrence of a specific event; cellular signal; chemical pathway; enzymatic reaction; cellular process; interaction between two or more entities; biological event; disease; disorder; or condition.

Initial loading dose: As used herein, an “initial loading dose” refers to a first dose of a therapeutic agent that may differ from one or more subsequent doses. Initial loading doses may be used to achieve an initial concentration of a therapeutic agent or level of activity before subsequent doses are administered.

Intravenous: As used herein, the term “intravenous” refers to the area within a blood vessel. Intravenous administration typically refers to delivery of a compound into the blood through injection in a blood vessel (e.g., vein).

In vitro: As used herein, the term “in vitro” refers to events that occur in an artificial environment (e.g., in a test tube or reaction vessel, in cell culture, in a Petri dish, etc.), rather than within an organism (e.g., animal, plant, or microbe).

In vivo: As used herein, the term “in vivo” refers to events that occur within an organism (e.g., animal, plant, or microbe or cell or tissue thereof).

Lactam bridge: As used herein, the term “lactam bridge” refers to an amide bond that forms a bridge between chemical groups in a molecule. In some cases, lactam bridges are formed between amino acids in a polypeptide.

Linker: The term “linker” as used herein refers to a group of atoms (e.g., 10-1,000 atoms), molecule(s), or other compounds used to join two or more entities. Linkers may join such entities through covalent or non-covalent (e.g., ionic or hydrophobic) interactions. Linkers may include chains of two or more polyethylene glycol (PEG) units. In some cases, linkers may be cleavable.

By “lower” or “reduce” in the context of a disease marker or symptom is meant a significant decrease in such level, often statistically significant. The decrease can be, for example, at least 10%, at least 20%, at least 30%, at least 40% or more, and is preferably down to a level accepted as within the range of normal for an individual without such disorder.

By “increase” or “raise” in the context of a disease marker or symptom is meant a significant rise in such level, often statistically significant. The increase can be, for example, at least 10%, at least 20%, at least 30%, at least 40% or more, and may be up to a level accepted as within the range of normal for an individual without such disorder.

Minute volume: As used herein, the term “minute volume” refers to the volume of air inhaled or exhaled from a subject's lungs per minute.

Non-proteinogenic: As used herein, the term “non-proteinogenic” refers to any non-natural proteins, such as those with non-natural components, such as non-natural amino acids.

Patient: As used herein, “patient” refers to a subject who may seek or be in need of treatment, requires treatment, is receiving treatment, will receive treatment, or a subject who is under the care of a trained professional for a particular disease or condition.

Any amino acid-based molecule (natural or non-natural) may be termed a polypeptide and this term embraces peptides, peptidomimetics, and proteins. Peptides are traditionally considered to range in size from about 4 to about 50 amino acids. Polypeptides larger than about 50 amino acids are generally termed proteins. C5 inhibitor polypeptides of the present disclosure may be peptidomimetics. A peptidomimetic or polypeptide mimetic is a polypeptide in which the molecule contains structural elements that are not found in natural polypeptides (i.e., polypeptides comprised of only the 20 proteinogenic amino acids).

Pharmaceutical composition: As used herein, the term “pharmaceutical composition” refers to a composition with at least one active ingredient (e.g., a C5 inhibitor) in a form and amount that permits the active ingredient to be therapeutically effective.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers to any ingredient other than active agents (e.g., active agent zilucoplan and/or active metabolites thereof or variants thereof) present in a pharmaceutical composition and having the properties of being substantially nontoxic and non-inflammatory in a patient. In some embodiments, a pharmaceutically acceptable excipient is a vehicle capable of suspending or dissolving the active agent. Excipients may include, for example: anti-adherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspending or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Plasma compartment: As used herein, the term “plasma compartment” refers to intravascular space occupied by blood plasma.

Salt: As used herein, the term “salt” refers to a compound made up of a cation with a bound anion. Such compounds may include sodium chloride (NaCl) or other classes of salts including, but not limited to acetates, chlorides, carbonates, cyanides, nitrites, nitrates, sulfates, and phosphates.

Sample: As used herein, the term “sample” refers to an aliquot or portion taken from a source and/or provided for analysis or processing. In some embodiments, a sample is from a biological source such as a tissue, cell or component part (e.g., a body fluid, including but not limited to blood, mucus, lymphatic fluid, synovial fluid, cerebrospinal fluid, saliva, amniotic fluid, amniotic cord blood, urine, vaginal fluid and semen). In some embodiments, a sample may be or include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, or organs. In some embodiments, a sample is or includes a medium, such as a nutrient broth or gel, which may contain cellular components, such as proteins. In some embodiments, a “primary” sample is an aliquot of the source. In some embodiments, a primary sample is subjected to one or more processing (e.g., separation, purification, etc.) steps to prepare a sample for analysis or other use.

Subcutaneous: As used herein, the term “subcutaneous” refers to the space underneath the skin. Subcutaneous administration is delivery of a compound beneath the skin.

Subject: As used herein, the term “subject” refers to any organism to which a compound or method in accordance with the disclosure may be administered or applied, e.g., for experimental, diagnostic, prophylactic, and/or therapeutic purposes. Typical subjects include animals (e.g., mammals such as mice, rats, rabbits, porcine subjects, non-human primates, and humans). In some applications, the subject is human.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” means an amount of an agent to be delivered (e.g., C5 inhibitor) that is sufficient, when administered to a subject suffering from or susceptible to a disease, disorder, and/or condition, to treat, improve symptoms of, diagnose, prevent, and/or delay the onset of the disease, disorder, and/or condition.

Tidal volume: As used herein, the term “tidal volume” refers to the normal lung volume of air displaced between breaths (in the absence of any extra effort).

Tₘₐₓ: As used herein, the term “Tₘₐₓ” refers to the time period for which maximum concentration of a compound in a subject or fluid is maintained.

Treating: As used herein, the term “treating” refers to partially or completely alleviating, ameliorating, improving, relieving, delaying onset of, inhibiting progression of, reducing severity of, and/or reducing incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Treatment dose: As used herein, “treatment dose” refers to one or more doses of a therapeutic agent administered in the course of addressing or alleviating a therapeutic indication. Treatment doses may be adjusted to maintain a desired concentration or level of activity of a therapeutic agent in a body fluid or biological system.

Volume of distribution: As used herein, the term “volume of distribution” or “V_{dist}” refers to a fluid volume required to contain the total amount of a compound in the body at the same concentration as in the blood or plasma. The volume of distribution may reflect the extent to which a compound is present in the extravascular tissue. A large volume of distribution reflects the tendency of a compound to bind to tissue components compared with plasma protein components. In a clinical setting, V_{dist} can be used to determine a loading dose of a compound to achieve a steady state concentration of that compound.

### V. Equivalents and Scope

While various embodiments of the invention have been particularly shown and described, it will be understood by those skilled in the art that various changes in form and details may be made therein without departing from the spirit and scope of the invention as defined by the appended claims.

Those skilled in the art will recognize or be able to ascertain using no more than routine experimentation, many equivalents to the specific embodiments in accordance with the invention described herein. The scope of the present invention is not intended to be limited to the above description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the terms “consisting of” and “or including” are thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### VI. Additional Embodiments

Notwithstanding the appended claims, this disclosure provides the embodiments defined by the following clauses:

Clause 1. A method of treating refractory generalized myasthenia gravis (gMG) in a human patient in need thereof, the method including: administering a therapeutically effective amount of zilucoplan to a human patient identified as (i) positive for auto-antibodies binding to nicotinic acetylcholine receptor (anti-AChR) and (ii) having refractory gMG.

Clause 2. The method of clause 1, where, prior to administration, the patient is refractory to treatment for 1 year or more with immunosuppressant therapy (IST) and requires chronic plasma exchange or chronic IVIG to maintain clinical stability.

Clause 3. The method of clause 1 or 2, where the patient experiences a reduction in Myasthenia Gravis Activities of Daily Living (MG-ADL) score of at least 3 points from baseline after 12 weeks of treatment (e.g., at least 4 points, at least 5 points, at least 6 points, at least 8, at least 10, or at least 12 points from baseline).

Clause 4. The method of any one of clauses 1 to 3, where the patient experiences reduction in MG-ADL score of at least 2 points from baseline after 8 weeks of treatment (e.g., at least 3 points, at least 4 points, at least 5 points, at least 6 points, at least 8, at least 10, or at least 12 points from baseline).

Clause 5. The method of any one of clauses 1 to 4, where the patient experiences a clinically meaningful improvement (reduction) in quantitative Myasthenia Gravis (QMG) score, in Myasthenia Gravis Composite (MGC) score, or in quality of life as measured by Myasthenia Gravis Quality of Life revised (MG-QOL-15r) score, after 12 weeks of treatment.

Clause 6. The method of clause 5, where the patient experiences a reduction in QMG score of at least 3 points from baseline after 12 weeks of treatment (e.g., at least 4 points, at least 5 points, at least 6 points, at least 8, at least 10, or at least 12 points from baseline).

Clause 7. The method of clause 6, where patient experiences a reduction in QMG score of at least 3 points from baseline after 8 weeks of treatment (e.g., at least 4 points, at least 5 points, at least 6 points, at least 8, at least 10, or at least 12 points from baseline).

Clause 8. The method of any one of clauses 3 to 7, where the patient experiences a reduction in MGC score of at least 3 points from baseline after 12 weeks of treatment (e.g., at least 4 points, at least 5 points, at least 6 points, at least 8, at least 10, or at least 12 points from baseline).

Clause 9. The method of any one of clauses 3 to 8, where the patient experiences a reduction in MG-QOL-15r score of at least 2-points from baseline after 12 weeks of treatment (e.g., at least 3 points, at least 4 points, at least 5 points, at least 6 points, at least 8, at least 10, or at least 12 points from baseline).

Clause 10. The method of any one of clauses 1 to 9, where zilucoplan administration is subcutaneous (SC).

Clause 11. The method of any one of clauses 1 to 10, where zilucoplan is administered daily.

Clause 12. The method of any one of clauses 1 to 11, where zilucoplan is administered to the patient for 12 weeks or more.

Clause 13. The method of clause 11 or 12, where zilucoplan is administered at a daily dose of from about 0.1 mg/kg (mg zilucoplan/kg subject body weight) to about 0.6 mg/kg.

Clause 14. The method of clause 13, where zilucoplan is administered at a daily dose of from about 0.1 mg/kg to about 0.3 mg/kg.

Clause 15. The method of clause 14, where zilucoplan is administered at a daily dose of 0.3 mg/kg.

Clause 16. The method of any of clauses 1 to 15, where zilucoplan is administered using a self-administration device.

Clause 17. The method of clause 16, where the self-administration device includes the prefilled syringe.

Clause 18. The method of clause 17, where the syringe is a glass syringe and includes a 29-gauge needle.

Clause 19. The method of any one of clauses 16 to 18, where the self-administration device has a maximum fill volume of at least 1 mL.

Clause 20. The method of clause 19, where the self-administration device includes a zilucoplan solution having a volume of from about 0.15 ml to about 0.81 mL.

Clause 21. The method of clause 20, where the solution includes from about 4 mg/ml to about 200 mg/mL zilucoplan.

Clause 22. The method of clause 21, where the solution includes about 40 mg/mL zilucoplan.

Clause 23. The method of any of clauses 16 to 22, where the self-administration device includes an aqueous solution of zilucoplan, or a sodium salt form thereof.

Clause 24. The method of clause 23, where the solution includes phosphate-buffered saline (PBS).

Clause 25. The method of clause 23 or 24, where the solution is preservative-free.

Clause 26. The method of any one of clauses 1 to 25, where the patient is between 18 and 85 years old.

Clause 27. The method of any one of clauses 1 to 26, where the patient does not need or receive rescue therapy during zilucoplan administration.

Clause 28. The method of any one of clauses 1 to 27, where zilucoplan administration is carried out at an MG disease stage that is prior to a critical or crisis stage of MG.

Clause 29. The method of any of clauses 1 to 28, where the patient simultaneously receives standard of care gMG therapy over the course of zilucoplan treatment.

Clause 30. The method of clause 29, where the standard of care gMG therapy includes one or more of cholinesterase inhibitor treatment, pyridostigmine treatment, corticosteroid treatment, and IST.

Clause 31. The method of clause 29 or 30, where the subject receives cholinesterase inhibitor treatment over the course of zilucoplan treatment.

Clause 32. The method of any one of clauses 1 to 31, further including co-administering to the patient a therapeutically effective amount of an additional therapeutic agent.

Clause 33. The method of clause 32, where the additional therapeutic agent is an immunosuppressive agent.

Clause 34. The method of clause 33, where the immunosuppressive agent is selected from azathioprine, cyclosporine, cyclosporine A, mycophenolate mofetil, methotrexate, tacrolimus, cyclophosphamide, and rituximab.

Clause 35. The method of clause 32, where the additional therapeutic agent includes an inhibitor of autoantibody-mediated tissue destruction.

Clause 36. The method of clause 35, where the inhibitor of autoantibody-mediated tissue destruction includes a neonatal Fc receptor (FcRN) inhibitor.

Clause 37. The method of clause 36, where administration of the FcRN inhibitor includes intravenous immunoglobulin (IVIG) treatment.

Clause 38. Zilucoplan for use in treating refractory generalized myasthenia gravis (gMG) in a human patient in need thereof, where the human patient is identified as (i) positive for auto-antibodies binding to nicotinic acetylcholine receptor (anti-AChR) and (ii) having refractory gMG.

Clause 39. Use of zilucoplan for the preparation of a medicament for the treatment of refractory generalized myasthenia gravis (gMG) in a human patient in need thereof, where the human patient is identified as (i) positive for auto-antibodies binding to nicotinic acetylcholine receptor (anti-AChR) and (ii) having refractory gMG.

### EXAMPLES

### Example 1. Preparation of Zilucoplan

Zilucoplan is produced through a 3-step process: linear sequence synthesis through amino acid coupling, cyclization by linking a lysine sidechain to an aspartic acid sidechain, and lastly, coupling a glutamic acid-ethylene glycol₂₄-palmitoyl moiety to a second lysine sidechain.

The zilucoplan polypeptide is synthesized using standard solid-phase Fmoc/tBu methods. The synthesis is performed on a Liberty automated microwave peptide synthesizer (CEM, Matthews NC) using standard protocols with Rink amide resin, although other automated synthesizers without microwave capability may also be used. All amino acids were obtained from commercial sources. The coupling reagent used is 2-(6-chloro-1-H-benzotriazole-lyl)-1,1,3,3,-tetramethylaminium hexafluorophosphate (HCTU) and the base is diisopropylethylamine (DIEA). Polypeptides are cleaved from resin with 95% TFA, 2.5% TIS and 2.5% water for 3 hours and isolated by precipitation with ether. The crude polypeptide is purified using reverse phase preparative HPLC on a C18 column, with an acetonitrile/water 0.1% TFA eluant gradient from 20%-50% over 30 min. Eluant fractions containing pure polypeptide are collected and lyophilized and polypeptide is analyzed by LC-MS.

Zilucoplan (SEQ ID NO: 1; CAS Number: 1841136-73-9) was prepared as a cyclic peptide containing 15 amino acids (4 of which are non-natural amino acids), with an acetylated N-terminus, and a C-terminal carboxylic acid. The C-terminal lysine of the core peptide has a modified side chain, forming a N-ε-(PEG24-γ-glutamic acid-N-α-hexadecanoyl) lysine reside. This modified side chain includes a polyethyleneglycol spacer (PEG24) attached to an L-γ glutamic acid residue that is derivatized with a palmitoyl group. Zilucoplan includes an intramolecular cyclization via a lactam bridge between the side-chains of L-Lysl and L-Asp6. All of the amino acids in zilucoplan are L-amino acids. Zilucoplan has a molecular weight of 3562.23 g/mol and a chemical formula of C₁₇₂H₂₇₈N₂₄O₅₅.

The final molecule is purified by reverse phase chromatography, exchanged into the sodium salt form, and subsequently dried. The drug substance is supplied as a lyophilized powder and stored at −20° C.

Zilucoplan is prepared as an aqueous solution for injection containing 40 mg/mL of zilucoplan in a sterile, preservative-free formulation of 50 mM sodium phosphate and 76 mM sodium chloride at a pH of 7.0. The resulting composition was used to prepare a medicinal product, in accordance with current Good Manufacturing Practices (cGMPs), the medicinal product including a pre-filled 1 ml glass syringe with a 29 gauge, ½ inch staked needle placed within a BD ULTRASAFE PLUS™ (BD, Franklin Lakes, NJ) self-administration device.

Dose strength variation, therefore, is accomplished by varying the syringe fill volume.

### Example 2. Zilucoplan Administration and Storage

Zilucoplan is administered by subcutaneous (SC) injection and the dose administered (dose volume) can be adjusted based on subject weight on a mg/kg basis. This is achieved using a set of fixed dose presentations aligned to a set of weight brackets. For the Phase 3 clinical studies, three of the previously tested strengths are used (16.6 mg, 23.0 mg and 32.4 mg). In total, human dosing supports a broad weight range of 43 to 109 kg. Subjects who present with a higher body weight (>109 kg) are accommodated on a case-by-case basis, in consultation with a medical monitor.

Zilucoplan is stored at 2° C. to 8° C. Once dispensed to subjects, zilucoplan is stored at controlled room temperature (20° C. to 25° C.) for up to 30 days and is protected from sources of excessive temperature fluctuations such as high heat or exposure to light. Storage of zilucoplan outside of room temperatures is preferably avoided. Zilucoplan may be stored for up to 4 months are permitted under these conditions.

### Example 3. Efficacy of Zilucoplan for Myasthenia Gravis Treatment in Phase 2 Clinical Study

In a Phase 2 clinical study of gMG (NCT03315130), both high and low dose zilucoplan treatments were shown to be effective. See e.g., PCT Publication No. WO2020/086506 (“Neurological Disease Treatment with Zilucoplan”), the disclosure of which is herein incorporated by reference in its entirety.

For convenience, the details of that clinical study are reproduced below.

A multicenter, randomized, double-blind, placebo-controlled study was carried out to evaluate zilucoplan safety, tolerability, and preliminary efficacy in treating subjects with gMG. In the study, subjects were randomized in a 1:1:1 ratio to receive daily SC doses of 0.1 mg/kg zilucoplan, 0.3 mg/kg zilucoplan, or matching placebo. Randomization was stratified based on screening Quantitative Myasthenia Gravis (QMG) score (≤17 versus ≥18).

The Main Portion of the study included a Screening Period of up to 4 weeks and a 12-week Treatment Period. During the Treatment Period, subjects returned to the clinic weekly for the first 2 visits (Day 8 and Day 15) after the Day 1 visit, followed by visits at Week 4 (Day 29), Week 8 (Day 57), and Week 12 (Day 84) to evaluate safety, tolerability, and preliminary efficacy. Additional assessments included quality of life (QOL) questionnaires, biomarker samples, pharmacokinetics, pharmacodynamics, and optional pharmacogenomics. Safety assessments included physical examinations, vital signs, ECGs, clinical laboratory tests, AEs, and immunogenicity.

Zilucoplan and the matching placebo were supplied as sterile, preservative-free, aqueous solutions prefilled into 1 mL glass syringes with 29 gauge, 12 inch, staked needles placed within self-administration devices. Fill volumes were adjusted based on subject weight range to achieve correct mg/kg dose range. Subjects were instructed to self-administer SC doses daily.

Doses of zilucoplan were determined by target dose and weight, accomplished using fixed dose by weight brackets. These brackets were grouped by body weight category such that each subject received no less than the target minimum dose to avoid sub-therapeutic dosing. For the 0.1 mg/kg dose, subjects received, at a minimum, a fixed dose of 0.1 mg/kg (range: 0.10 to 0.14 mg/kg). For the 0.3 mg/kg dose, subjects received a minimum dose of 0.3 mg/kg (range: 0.30 to 0.42 mg/kg). Table 1 summarizes the dose presentations for zilucoplan 0.1 and 0.3 mg/kg doses. Subjects who presented with a higher body weight (>150 kg) were accommodated on a case-by-case basis. Matching placebo was provided in 2 presentations, 0.220 mL for the 0.1 mg/kg dose and 0.574 mL for the 0.3 mg/kg dose.

TABLE 1
Zilucoplan dose presentations by weight bracket

| Target | Dose Presentation | | Weight | Dose |
|---|---|---|---|---|
| Dose | Fill Volume | Dose | Range | Range |
| mg/kg | (mL) | (mg) | (kg) | mg/kg |
| 0.1 | 0.150 | 6 | ≥43 to <61 | 0.10 to 0.14 |
| 0.1 | 0.220 | 8.8 | ≥61 to <88 | 0.10 to 0.14 |
| 0.1 | 0.310 | 12.4 | ≥88 to <109 | 0.11 to 0.14 |
| 0.3 | 0.416 | 16.6 | ≥43 to <56 | 0.30 to 0.39 |
| 0.3 | 0.574 | 23 | ≥56 to <77 | 0.30 to 0.41 |
| 0.3 | 0.810 | 32.4 | ≥77 to ≤150 | 0.30 to 0.42 |

### Screening

Screening was carried out to determine subject study eligibility. Screening included QMG score assessment. The patient population most appropriate for zilucoplan treatment was expected to have a QMG score ≥12 when assessed at screening and baseline (off acetylcholinesterase inhibitor therapy, e.g., pyridostigmine, for at least 10 hours) with ≥4 test items scored at ≥2. Other eligibility criteria assessed during screening included age between 18 and 85; gMG diagnosis [according to Myasthenia Gravis Foundation of America (MGFA) criteria; Class II-IVa] at time of screening; positive serology for AChR autoantibodies; no change in corticosteroid dose for at least 30 days prior to baseline or anticipated to occur during the 12-week Treatment Period; and no change in immunosuppressive therapy, including dose, for at least 30 days prior to baseline or anticipated to occur during the 12-week Treatment Period. Female subjects of childbearing potential needed to have a negative serum pregnancy test at screening and a negative urine pregnancy test within 24 hours prior to the first dose of study drug, sexually active female subjects of childbearing potential (i.e., women who were not postmenopausal or who had not had a hysterectomy, bilateral oophorectomy, or bilateral tubal ligation) and all male subjects (who had not been surgically sterilized by vasectomy) agreed to use effective contraception during the study.

During screening, assessments were performed that included review of medical history and demographics, including collection of disease history with diagnosis of gMG according to MGFA criteria (Class II-IVa); serology for AChR autoantibodies; QMG score assessment; height and weight measurement; assessment of vital signs [heart rate (HR), body temperature, and blood pressure in the sitting position]; 12-lead ECG; assessment of prior Neisseria meningitidis vaccination; collection of blood samples for laboratory testing [hematology, chemistry, coagulation, adenosine deaminase (ADA) testing, and pharmacogenomic analysis]; collection of urine samples for urinalysis; and serum pregnancy testing for females of childbearing potential.

Subjects meeting any of the following criteria were excluded from the study: (1) thymectomy within 6 months prior to baseline or scheduled to occur during the 12-week Treatment Period; (2) abnormal thyroid function as determined by local standard; (3) known positive serology for muscle-specific kinase (MuSK) or lipoprotein receptor-related peptide 4 (LRP4); (4) Minimal Manifestation Status (MMS) of myasthenia gravis based on the clinical evaluation; (5) calculated glomerular filtration rate of <60 mL/min/1.73 m² based on the Modification of Diet in Renal Disease (MDRD) equation at Screening:

$$GRF ( \frac{mL / min}{1.73 {m}^{2}} ) = 175 \times {( {S}_{cr} )}^{- 1.154} \times {( Age )}^{- 0.203} \times ( 0.742 if female ) \times ( 1.212 if American American ) ;$$

(6) elevated liver function tests (LFTs) defined as total bilirubin or transaminases [aspartate aminotransferase (AST)/alanine aminotransferase (ALT)]>2 times the upper limit of normal (×ULN); (7) history of meningococcal disease; (8) current or recent systemic infection within 2 weeks prior to baseline or infection requiring IV antibiotics within 4 weeks prior to baseline; (9) pregnant, planning to become pregnant, or nursing female subjects; (10) recent surgery requiring general anesthesia within 2 weeks prior to screening or surgery expected to occur during screening or the 12-week Treatment Period; (11) treatment with an experimental drug or another complement inhibitor within 30 days or 5 half-lives of the experimental drug (whichever is longer) prior to baseline; (12) treatment with rituximab within 6 months prior to baseline; (13) ongoing treatment with IV immunoglobulin G (IVIG) or plasma exchange (PLEX) or treatment within 4 weeks prior to baseline; (14) active neoplasm (other than benign thymoma) requiring surgery, chemotherapy, or radiation within the prior 12 months (subjects with a history of malignancy who have undergone curative resection or otherwise not requiring treatment for at least 12 months prior to screening with no detectable recurrence are allowed); (15) fixed weakness (‘burnt out’ MG) based on clinical judgement; (16) history of any significant medical or psychiatric disorder that render subject unsuitable for participation in the study; and (17) participation in another concurrent clinical trial involving an experimental therapeutic intervention (participation in observational studies and/or registry studies is permitted).

### Treatment Period

Randomized subjects received 0.1 mg/kg zilucoplan, 0.3 mg/kg zilucoplan, or matching placebo administered SC at the Day 1 visit. Following in-clinic education and training, all subjects self-injected daily SC doses of blinded study drug, according to randomized treatment allocation, for the subsequent 12 weeks. An injection device was provided for use during the study. Subjects were expected to remain on stable doses of standard of care (SOC) therapy for gMG throughout the study, including pyridostigmine, corticosteroids, or immunosuppressive drugs. Dosing on study visit days was withheld until QMG scoring and blood collection [for pharmacokinetic (PK) and pharmacodynamic (PD) analysis] was completed. On days when rescue therapy was concurrently administered, study drug dosing was held until after administration of rescue therapy and PK/PD sampling. Rescue therapy involved escalation of gMG therapy due to deterioration of subject clinical status. During rescue therapy, subjects received immunoglobulin (IVIG) or plasma exchange treatment.

During the Main Portion of the study, the total duration of study participation for all subjects was up to approximately 16 weeks, including a Screening Period of up to 4 weeks and a 12-week Treatment Period. A study Extension Portion was made available for continued zilucoplan administration.

Subjects received treatment with 0.1 mg/kg zilucoplan, 0.3 mg/kg zilucoplan, or matching placebo, according to randomization, from Day 1 to Day 84 during the Treatment Period of the Main Portion of the study. Subjects who completed the Day 84 visit (including those randomized to the placebo arm) had the option to continue treatment with zilucoplan in the Extension Portion of the study.

End of Study and Final Study procedures included weight measurement; review and documentation of concomitant medications; symptom-directed physical examination; assessment of vital signs (e.g., heart rate, body temperature, and blood pressure in sitting position); 12-lead ECG; collection of blood samples for laboratory testing (hematology, chemistry, coagulation, ADA testing, pharmacokinetic analysis, pharmacodynamic analysis, and biomarker analysis); collection of urine for urinalysis; urine pregnancy testing for females of childbearing potential; QMG score assessment; and assessment of MG-ADL, MG-QOL15r, and MG composite (MGC).

### Sample Analysis

During the Main Portion of the study, blood samples for PK and PD analysis were collected from all subjects at the time points presented in Table 2. Extension Portion blood sample collection for PK and PD analysis was designed to be identical under rescue therapy scenarios. Additionally, blood taken at Week 36 of the Extension Portion was planned to follow the “Day 1” schedule described for Main Portion blood collection.

TABLE 2
Time points for blood collections

| | | During Rescue Therapy | |
|---|---|---|---|
| | | | At sites where |
| | | At sites where | rescue therapy |
| | | rescue therapy | is NOT |
| | | is administered | administered |
| Day 1 | Day 84 | locally | locally |
| Pre-dose | Pre-dose (any | Within | Prior to |
| (within 1 hour | time prior to | 1 hour before | administration |
| before first | Day 84 | administration | of the first |
| dose of | study drug | of rescue therapy | course of |
| study drug) | administration) | | rescue therapy |
| 1-hour | 1-hour | For PK (for | For PK: |
| post-dose | post-dose | plasma exchange | Prior to |
| (±30 minutes) | (±30 minutes) | only): Measured | administration |
| | | in the exchanged | of the first |
| | | plasma | course of |
| | | For PD: Within | rescue therapy |
| | | 1 hour before | For PD: After |
| | | administration | administration |
| | | of rescue therapy | of the last |
| | | | course of |
| | | | rescue therapy |
| 3 hours | | Within | After |
| post-dose | | 1 hour after | administration |
| (±30 minutes) | | administration | of the last |
| 6 hours | | of rescue therapy | course of |
| post-dose | | | rescue therapy |
| (±90 minutes) | | | |

On all other study visit days, single PK and PD samples were collected prior to administration of study drug. Plasma concentrations of zilucoplan and its metabolites were measured in all blood samples.

Blood samples for safety analyses were collected at the following time points on Day 1: (i) pre-dose (within 1 hour before first dose administration of study drug) and (ii) 6 hours post-dose (±90 minutes). At all other study visits, samples for safety analysis were collected prior to administration of study drug. An additional blood sample for testing was taken at 6 hours post-dose (±90 minutes) on Day 84 from subjects intending to participate in the Extension Portion of the study. Blood sample analytes assessed included those listed in Table 3.

TABLE 3
Blood sample analytes
Analyte

Alanine aminotransferase (ALT)
Albumin
Alkaline phosphatase (ALP)
Amylase
Aspartate aminotransferase (AST)
Bicarbonate
Bile acids
Bilirubin (total, direct, and indirect)
Blood urea nitrogen (BUN)
Calcium
Chloride
Creatinine
Gamma-glutamyl transferase (GGT)
Glucose
Lipase
Potassium
Sodium
Total protein
Uric acid
Hematocrit
Hemoglobin
Mean corpuscular volume (MCV)
Platelet count
White blood cell (WBC) count and differential (%)
International normalized ratio (INR)/prothrombin time (PT)
Fibrinogen
Partial thromboplastin time (PTT) or activated partial
thromboplastin time (aPTT)
C-reactive protein (CRP)
Creatine phosphokinase (CPK)

MG pathophysiology biomarker analysis [e.g., complement fixation, complement function, complement pathway proteins, autoantibody characterization (titer and immunoglobulin class), and inflammatory markers] was available to provide further insight into clinical efficacy and safety of zilucoplan in subjects with gMG. Assessment of complement protein levels and complement activity can be used to evaluate response to zilucoplan and to understand subject characteristics related to variations in drug response. Inflammation marker testing can be used to assess correlation with complement function and clinical response to zilucoplan. A list of analytes can be created through review of the literature, ongoing clinical studies, and ongoing exploratory work and finalized after completion of the study.

The primary efficacy endpoint was the change from baseline to Week 12 (Day 84) in QMG score. The QMG score is a standardized and validated quantitative strength scoring system that was developed specifically for gMG and has been used previously in clinical trials. Higher scores are representative of more severe impairment. Recent data suggest that improvements in the QMG score of 2 to 3 points may be considered clinically meaningful, depending upon disease severity [Barohn, R J et al. 1998, Ann N Y Acad Sci. 841:769-72; Katzberg, H D et al. 2014, Muscle Nerve, 49(5):661-5]. QMG assessment was performed at each study visit and at screening to assess subject eligibility. The QMG assessment was performed at approximately the same time of day (preferably in the morning) at each visit throughout the study. If a subject was receiving a cholinesterase inhibitor (e.g., pyridostigmine), the dose was withheld for at least 10 hours prior to QMG test. 0.3 mg/kg and 0.1 mg/kg dose groups were compared to placebo dose group and linear trends were assessed based on all three treatment groups.

Secondary efficacy endpoints included Week 12 change from baseline in MG-ADL, MG-QOL15r, and MG Composite. Each of the active doses was compared to the placebo group. For subjects with ≥3-point reduction in QMG score at Week 12 and subjects requiring rescue therapy over the 12-week Treatment Period, the rate of subjects meeting the endpoint for each of the active treatment groups was compared to the placebo group.

The specific order of efficacy endpoint analyses was arranged to reduce subject fatigue and enhance result reliability. MG-QOL15r analysis was conducted first, followed by MG-ADL analysis, QMG score assessment, and MG Composite. The same evaluator was used throughout the study to reduce assessment variability.

### Results

Study results from a broad, demographically well balanced population of participants were obtained. Pre-study baseline characteristics for study participants are presented in Table 4. In the Table, “SD” refers to “standard deviation” and “SOC” refers to “standard of care.”

TABLE 4
Baseline study participant characteristics

| | | | 0.1 mg/kg | | 0.3 mg/kg | |
|---|---|---|---|---|---|---|
| | Placebo | | zilucoplan | | zilucoplan | |
| Characteristic | (n = 15) | | (n = 15) | | (n = 14) | |
| Age, years, mean (SD) | 48.4 | (15.7) | 45.5 | (15.7) | 54.6 | (15.5) |
| Male, n (%) | 4 | (26.7%) | 7 | (46.7%) | 10 | (71.4%) |
| Weight, kg, mean (SD) | 85.27 | (21.44) | 93.71 | (24.72) | 110.9 | (30.79) |
| BMI, mean (SD) | 30.856 | (7.386) | 32.804 | (6.548) | 36.000 | (8.242) |
| Race, n (%) | | | | | | |
| White | 12 | (80.0%) | 13 | (86.7%) | 11 | (78.6%) |
| Asian | 1 | (6.7%) | 0 | | 1 | (7.1%) |
| Black or African American | 2 | (13.3%) | 2 | (13.3%) | 2 | (14.3%) |
| MGFA Class at Screening | | | | | | |
| II | 7 | (46.7%) | 5 | (33.3%) | 5 | (35.7%) |
| III | 8 | (53.3%) | 10 | (66.7%) | 5 | (35.7%) |
| IV | 0 | | 0 | | 4 | (28.6%) |
| Age at Disease Onset, years, mean (SD) | 40.3 | (17.79) | 37.3 | (16.04) | 46.9 | (19.48) |
| Duration of Disease, years, mean (SD) | 8.04 | (0.1, 20.9) | 8.71 | (1.6, 24.1) | 8.3 | (0.5, 26.0) |
| Baseline QMG Score, mean (SD) | 18.7 | (4.0) | 18.7 | (4.0) | 19.1 | (5.1) |
| Baseline MG-ADL Score, mean (SD) | 8.8 | (3.6) | 6.9 | (3.3) | 7.6 | (2.6) |
| Baseline MGC Score, mean (SD) | 18.7 | (5.7) | 14.5 | (6.3) | 14.6 | (6.3) |
| Baseline MG-QOL15r Score, mean (SD) | 15.9 | (7.4) | 19.1 | (5.0) | 16.5 | (7.3) |
| Prior MG Therapies (Standard of Care) | | | | | | |
| Pyridostigmine, n (%) | 14 | (93.3%) | 15 | (100%) | 14 | (100%) |
| Corticosteroids, n (%) | 13 | (86.7%) | 13 | (86.7%) | 14 | (100%) |
| Immunosuppressants, n (%) | 12 | (80.0%) | 12 | (80.0%) | 9 | (64.3%) |
| Prior IVIG, n (%) | 9 | (60.0%) | 8 | (53.3%) | 10 | (71.4%) |
| Prior Plasma Exchange, n (%) | 7 | (46.7%) | 9 | (60.0%) | 7 | (50.0%) |
| Prior Thymectomy, n (%) | 5 | (33.3%) | 8 | (53.3%) | 7 | (50.0%) |
| Prior MG crisis requiring intubation, n (%) | 3 | (20.0%) | 4 | (26.7%) | 2 | (14.3%) |

The population included subjects with baseline disease characteristics indicative of refractory as well as non-refractory disease status. Baseline disease characteristics including MGFA classification and efficacy outcome measures were also well balanced among study participants. In the study, 15 subjects received placebo, while 15 subjects received low dose zilucoplan (0.1 mg/kg) and 14 subjects received high dose zilucoplan (3 mg/kg). Significance testing was pre-specified at a 1-sided alpha of 0.1.

Baseline demographic characteristics were similar across groups with respect to mean age (48.4 to 54.6 years years), race representation (78.6% to 86.7% white), mean weight (85.27 to 110.9 kg) and mean BMI (30.856 to 36.000). There was an imbalance between the groups with respect to gender 71.4%, 46.7%, and 26.7% male in the 0.3 mg/kg zilucoplan, 0.1 mg/kg zilucoplan, and placebo groups, respectively. However, gender is not known to play a significant role in treatment response in gMG.

Medical history including duration of disease, prior MG crisis, prior thymectomy, and prior treatment with pyridostigmine, corticosteroid therapy, immunosuppressive agents, or rescue therapy with IVIG or PLEX was well balanced across treatment groups. Over 90% of subjects in each group had received acetylcholinesterase inhibitors; over 85% had received corticosteroids; 64.3 to 80% had received immunosuppressive therapy; 53.3 to⁷1.4% had received IVIG; and 46.7 to 60.0% had undergone plasma exchange.

MG disease severity as measured by MGFA classification was similar across the treatment groups with all subjects in the 0.1 mg/kg zilucoplan and placebo groups being in MGFA class II (mild disease severity) and III (moderate disease severity), although the 0.3 mg/kg zilucoplan group also included four subjects in MGFA class IV (severe disease).

MG specific baseline characteristics were well balanced across the primary (QMG) and first secondary (MG-ADL) endpoint scores, with mean baseline QMG scores of 19.1, 18.7, and 18.7; and mean MG-ADL scores of 7.6, 6.9, and 8.8 in the 0.3 mg/kg zilucoplan, 0.1 mg/kg zilucoplan, and placebo groups, respectively. The MG-QOL15r was approximately three points higher in the 0.1 mg/kg zilucoplan group than in the 0.3 mg/kg zilucoplan group with mean MG-QOL15r scores of 16.5, 19.1, and 15.9 in the 0.3 mg/kg zilucoplan, 0.1 mg/kg zilucoplan, and placebo groups, respectively. The MGC was >4 points higher in the placebo group than in the other two groups with mean MGC scores of 14.6, 14.5, and 18.7 in the 0.3 mg/kg zilucoplan, 0.1 mg/kg zilucoplan, and placebo groups, respectively.

Clinical efficacy outcomes are provided in Table 5. In the Table, P-values are one-sided based on analysis of covariance (ANCOVA) model, with baseline values as covariates and using last observation carried forward (LOCF) for subjects who received rescue therapy. “LS” refers to “least squares,” “CFB” refers to change from baseline, and “se” refers to “standard error.”

TABLE 5
Clinical efficacy outcomes

| | | MG- | MG- | |
|---|---|---|---|---|
| Value | QMG | ADL | QOL15r | MGC |
| 0.3 mg/kg zilucoplan | −6.0 (1.2) | −3.4 (0.9) | −5.9 (1.7) | −7.4 (1.6) |
| CFB LS mean(se) | | | | |
| 0.1 mg/kg zilucoplan | −5.5 (1.2) | −3.3 (0.9) | −7.4 (1.7) | −5.3 (1.5) |
| CFB LS mean (se) | | | | |
| Placebo LS mean | −3.2 (1.2) | −1.1 (0.9) | −2.1 (1.7) | −3.3 (1.6) |
| CFB (se) | | | | |
| 0.3 mg/kg zilucoplan | −2.8 (1.7) | −2.3 (1.3) | −3.7 (2.4) | −4.1 (2.2) |
| CFB LS mean | | | | |
| difference vs. placebo | | | | |
| p- value | 0.05 | 0.04 | 0.06 | 0.04 |
| 0.1 mg/kg zilucoplan | −2.3 (1.7) | −2.2 (1.3) | −5.3 (2.4) | −2.0 (2.2) |
| CFB LS mean | | | | |
| difference vs. | | | | |
| placebo (se) | | | | |
| p- value | 0.09 | 0.05 | 0.02 | 0.19 |

0.3 mg/kg treatment groups showed clinically meaningful and statistically significant improvement in QMG score (≥3 points) over baseline at the 12-week time point, with mean difference over placebo of −2.8 (p=0.05). Clinically meaningful and statistically significant improvement in MG-ADL score (≥2 points) over baseline was also observed with this treatment group at week 12, with mean difference over placebo of −2.3 (p=0.04). Clinically meaningful and statistically significant improvements were also observed in the low dose treatment group, demonstrating only slightly lower changes from baseline than those observed with higher dose subjects. With lower dose treatment (0.1 mg/kg), clinically meaningful and statistically significant improvement in QMG score was observed with −2.3 point mean difference (p=0.09) over placebo at week 12. Clinically meaningful and statistically significant changes in MG-ADL score at week 12 were also observed with this group (−2.2 mean difference over placebo; p=0.05). Changes in MG-ADL from baseline for placebo versus mean changes of the pooled low and high dose zilucoplan treatment groups (n=29) show statistically significant advantage for zilucoplan treatment over placebo (−2.2 mean difference over placebo; p=0.047, 2-sided). In comparing treatment responders to placebo responders in the high dose group to placebo at 12 weeks, the highest levels of improvements in QMG score and MG-ADL were all in the zilucoplan treatment group and there were generally more patients on zilucoplan improving at each cut-off level compared to placebo.

Zilucoplan reduced the need for rescue treatment with only one subject (7%) in the low dose treatment group and zero subjects in the high dose treatment group requiring rescue (as compared to three subjects (20%) requiring rescue therapy in the placebo group). No significant endpoint differences were observed between treatment groups based on prior therapy covariates (immunosuppressive therapy, IVIG, or PLEX), all with P values above 0.20.

Responder analysis was conducted for QMG and MG-ADL endpoints. A clinically meaningful response on the QMG total score was defined as an improvement of three points or more, in line with the higher end of the established minimal clinically important difference (MCID) for QMG (Barohn et al. 1998; Katzberg et al. 2014). The proportion of responders at week 12 using the cut-off of ≥3 points on QMG was higher for subjects receiving 0.3 mg/kg zilucoplan (n=10/14) and 0.1 mg/kg zilucoplan (n=10/15) compared to the placebo (n=8/15) group. Additional pre-planned analyses showed an advantage for the zilucoplan treated groups at all cut-offs on QMG, including no subject with worsening in the 0.3 mg/kg zilucoplan group compared to three and two subjects in the 0.1 mg/kg zilucoplan and placebo groups, respectively. None of these differences were statistically significant except for the 0.1 mg/kg zilucoplan group vs. placebo at the ≥7 points and ≥11 points improvement cut-offs (Table 6), no correction for multiple testing was performed. Overall, the data were consistent with the primary analysis, generally showing higher clinical response in the zilucoplan treated arms than in the placebo group.

TABLE 6
Proportion of subjects with improved/worsened
QMG scores at week 12

| | 0.3 mg/kg | | 0.1 mg/kg | | | |
|---|---|---|---|---|---|---|
| | zilucoplan | | zilucoplan | | Placebo | |
| Cut-off | n = 14 | | n = 15 | | n = 15 | |
| Worsened | 0 | (0%) | 3 | (20.0%) | 2 | (13.3%) |
| ≥3 points improvement | 10 | (71.4%) | 10 | (66.7%) | 8 | (53.3%) |
| ≥7 points improvement | 6 | (42.9%) | 8 | (53.3%)* | 3 | (20.0%) |
| ≥11 points improvement | 2 | (14.3%) | 4 | (26.7%)* | 0 | (0%) |

*p < 0.1 (Fischer's exact test, one-sided p-value comparison of each zilucoplan dose group versus placebo).

The generally accepted MCID on the MG-ADL total score is an improvement of two points or more (Wolfe et al. 1999; Muppidi et al. 2011). Analyses also included higher cut-offs up to a difference of at least 11 points. The proportion of responders at week 12 using the cut-off of ≥2 points on MG-ADL was higher for subjects receiving zilucoplan at 0.3 mg/kg (n=10/14, 71.4%) and 0.1 mg/kg (n=10/15) compared to the placebo (n=8/15) group (Table 7).

TABLE 7
Proportion of subjects with improved/worsened
MG-ADL scores at week 12

| | 0.3 mg/kg | | 0.1 mg/kg | | |
|---|---|---|---|---|---|
| | zilucoplan | | zilucoplan | | Placebo |
| Cut-off | n = 14 | | n = 15 | | n = 15 |
| Worsened | 1 | (7.1%)* | 1 | (6.7%)* | 5 (33.3%) |
| ≥2 points improvement | 10 | (71.4%) | 9 | (60.0%) | 8 (53.3%) |

*p < 0.1 (Fischer's exact test, one-sided p, no correction for multiple testing).

A minimal symptom expression (MSE) endpoint was assessed to determine how many subjects become free or virtually free of gMG symptoms (based on achieving an MG-ADL total score of 0 or 1) with zilucoplan therapy. In this study 35.7% (5/14) subjects in the 0.3 mg/kg zilucoplan group achieved an MG-ADL of 0 or 1, compared to 26.7% (4/15) in the 0.1 mg/kg zilucoplan and 13.3% (2/15) in the placebo group. Further, the percent achievement for the high dose treatment group was greater than that observed with 26 weeks eculizumab treatment (based on eculizumab study results presented in Vissing, J. et al., 2018. AANEM Abstract 193). This analysis underscored the large extent to which improvement of subjective perception of disease burden can be achieved within a short period of time with zilucoplan. The dose response with a higher proportion of patients achieving MSE in the 0.3 mg/kg zilucoplan group was again evident in this analysis.

Sparse sampling was performed for assessment of pharmacokinetic and pharmacodynamic data. Steady state zilucoplan plasma concentrations were achieved within the first two weeks of treatment, and no further accumulation was observed. Trough levels of zilucoplan at steady state (at two weeks or later) in the 0.3 mg/kg zilucoplan dose group ranged between 7,168 ng/mL and 13,710 ng/mL while they were between 2,364 ng/mL and 7,290 ng/mL in the 0.1 mg/kg zilucoplan dose group. 0.3 mg/kg zilucoplan dose consistently achieved complete terminal complement pathway inhibition as measured by sheep red blood cell (sRBC) hemolysis assay (≥95% inhibition at trough). The 0.1 mg/kg dose of zilucoplan, by contrast, did not consistently achieve complete hemolysis inhibition. Pharmacokinetic and pharmacodynamic results were closely correlated in patients with gMG, indicating complete inhibition of the terminal complement pathway with zilucoplan plasma concentrations above approximately 9,000 ng/ml.

Overall, both high and low dose zilucoplan treatments were effective, with a favorable safety profile, and higher doses yielding more robust clinical improvement.

### Extension Portion

At the conclusion of the Treatment Period in the Main Portion of the Phase 2 study described in the above Example, all subjects were given the option to receive zilucoplan in an Extension Portion of the study provided they met Extension Portion selection criteria. Subjects assigned to a zilucoplan treatment arm during the Main Portion of the study continued to receive the same dose of study drug during the Extension Portion. Subjects assigned to the placebo arm during the Main Portion of the study were randomized in a 1:1 ratio to receive daily SC doses of 0.1 mg/kg zilucoplan or 0.3 mg/kg zilucoplan. Assessments and visits during the first 12 weeks of the Extension Portion were identical to the Main Portion of the study for all subjects to ensure appropriate monitoring of subjects transitioning from placebo to active treatment and to maintain blinding of treatment assignment.

Selection criteria for inclusion in the Extension Portion included: (1) positive serology for AChR autoantibodies; (2) negative serum pregnancy test at screening for female subjects of childbearing potential and a negative urine pregnancy test within 24 hours prior to the first dose of study drug; (3) agreement to use effective contraception during the study for sexually active female subjects of childbearing potential (i.e., women who are not postmenopausal or who have not had a hysterectomy, bilateral oophorectomy, or bilateral tubal ligation) and all male subjects (who have not been surgically sterilized by vasectomy); (4) use of any disallowed medications per the exclusion criteria from the Main Portion of the study or altered dosing of any other concomitant medication, unless medically indicated; (5) and no new medical conditions since entry into the Main Portion of the study.

During the Extension Portion of the study, biopsy sections obtained from subjects undergoing thymectomy, lymphadenectomy, or other surgical excision were sent for exploratory immunohistochemical and biomarker analysis.

41 patients completed the 12-week Extension Portion of the study (24 weeks total treatment period). Sustained responses were observed for each of: (1) QMG, 8.7 point reduction over baseline, p<0.0001; (2) MG-ADL, 4.5 point reduction over baseline, p<0.0001; (3) MG Composite, 10.2 point reduction over baseline, p<0.0001; and MG-QOL15r, 7.5 point reduction over baseline, p=0.0006.

Placebo subjects crossing over to active drug after 12 weeks also experienced rapid, clinically meaningful, and statistically significant improvement for each endpoint: (1) QMG, 3.1 point reduction over pretreatment level (level associated with 12 week placebo treatment), p=0.01; (2) MG-ADL, 3.6 point reduction over pretreatment level, p=0.0004; (3) MG Composite, 5.5 point reduction over pretreatment level, p=0.004; and MG-QOL15r, 4.0 point reduction over pretreatment level, p=0.04.

### Example 4. Efficacy of Zilucoplan in RAISE Phase 3 Clinical Study

RAISE (also referred to as MG0010) (NCT04115293) is a Phase 3, multicenter, randomized, double-blind, placebo-controlled study of the Safety, Tolerability, and Efficacy of zilucoplan in subjects with generalized myasthenia gravis. The study involvedzilucoplan daily subcutaneous treatment versus placebo over 12-weeks to evaluate efficacy of zilucoplan in subjects with gMG, including both patients having refractory gMG, and patients having non-refractory gMG.

Dosage Regimen: 0.3 mg/kg zilucoplan daily subcutaneous administration for 12 weeks, with option for extension.

### Study Description:

Study participants were randomized in a 1:1 ratio to receive daily SC doses of ZLP 0.3 mg/kg or placebo. Randomization was stratified based on the Baseline MG-ADL Score (≤9 versus ≥10), Baseline QMG Score (≤17 versus ≥18), and geographical region (North America, Europe, and East Asia).

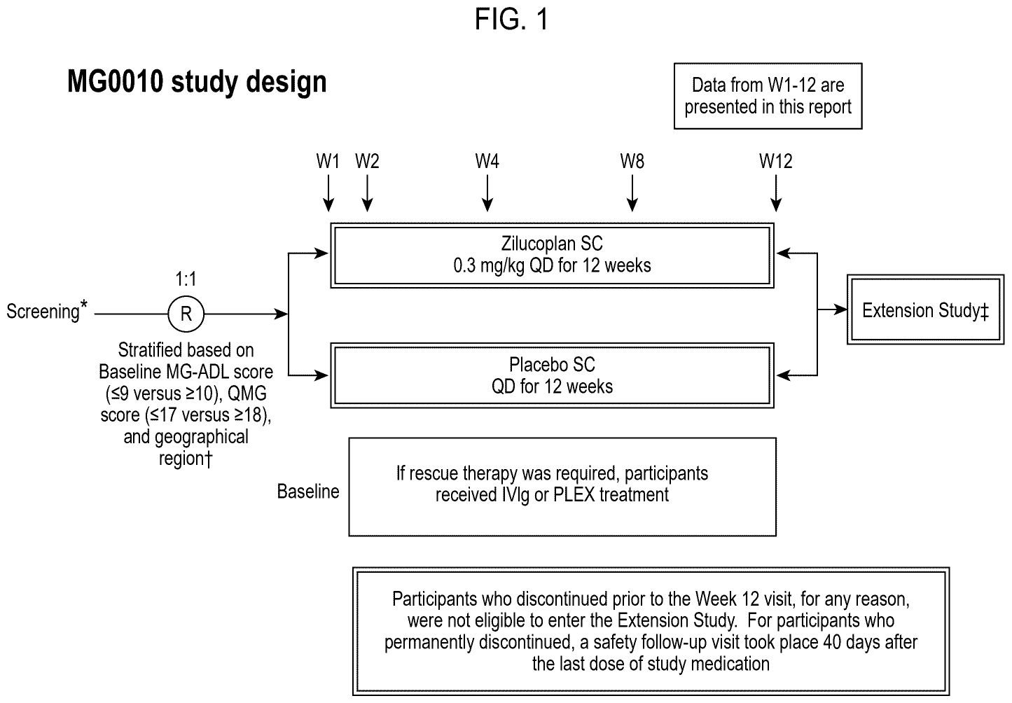

A schematic of this Phase 3 study design is presented in FIG. 1. The study included a Screening Period of up to 4 weeks and a 12 week double blind Treatment Period. During the double-blind Treatment Period, study participants returned to the clinic at Week 1, Week 2, Week 4, Week 8, and Week 12 to evaluate efficacy, safety, and tolerability.

After the 12-week double-blind, placebo-controlled period, all subjects have the option of receiving zilucoplan in an open label study extension. Subjects are evaluated for changes in MG-ADL (primary endpoint), QMG, MG Composite, and MG-QOL15r (key secondary endpoints). Primary and secondary endpoints are at Week 12 (at the end of the double-blind period) but the subjects were assessed during and after main study and extension study treatments.

Safety assessments included physical examinations, vital signs, ECG, clinical laboratory tests, AE monitoring, immunogenicity and the Columbia Suicide Severity Rating Scale.

All SOC therapy medications for gMG were to be kept at the same dose throughout the 12 week study, including corticosteroids and immunosuppressant therapy drugs. If escalation of gMG therapy (ie, “rescue therapy”) becomes necessary due to major deterioration of a study participant's clinical status, or risk of MG crisis as per the investigator's judgment, the study participant may receive IVIG or PLEX treatment as ‘rescue therapy’.

### Selected Inclusion Criteria:

Male or female aged ≥18 and <75 years;
Diagnosis of gMG [Myasthenia Gravis Foundation of America (MGFA) Class II-IV] at Screening;
Positive serology for acetylcholine receptor (AChR) autoantibodies;
MG-ADL Score of ≥6 at Screening and Baseline;
QMG score ≥12 at Screening and Baseline (off acetylcholinesterase inhibitor therapy for at least 10 hours) with 4 or more of the QMG test items scored at ≥2 at Screening and Baseline;
No change in corticosteroid dose for at least 30 days prior to Baseline or anticipated to occur during the 12-week Treatment Period;
No change in immunosuppressive therapy, including dose, for at least 30 days prior to Baseline or anticipated to occur during the 12-week Treatment Period; and
Patients immunized with meningococcal vaccines at least 2 weeks prior to administering first dose.

### Exclusion Criteria:

Thymectomy within 12 months prior to Baseline or scheduled to occur during the 12 week Treatment Period.
History of meningococcal disease.
Current or recent systemic infection within 2 weeks prior to Baseline or injection requiring intravenous (IV) antibiotics within 4 weeks prior to Baseline.

Patient demographics and baseline disease characteristics were generally balanced between treatment arms of the study (Table 8).

TABLE 8
Patient demographics and baseline disease characteristics

| | | | | Zilucoplan | |
|---|---|---|---|---|---|
| | | Placebo | | 0.3 mg/kg | |
| | | (N = 88) | | (N = 86) | |
| Aqe, years, mean (SD) | | 53.3 | (15.7) | 52.6 | (14.6) |
| Sex, male, n (%) | | 41 | (46.6) | 34 | (39.5) |
| Weight, kq, mean (SD) | | 88.2 | (26.6) | 90.1 | (22.9) |
| Geographic | North America | 46 | (52.3) | 45 | (52.3) |
| region, n (%) | Europe | 33 | (37.5) | 34 | (39.5) |
| | East Asia | 9 | (10.2) | 7 | (8.1) |
| MGFA disease | II (IIa, IIb) | 27 | (30.7) | 22 | (25.6) |
| class, n (%) | III (IIIa, IIIb) | 57 | (64.8) | 60 | (69.8) |
| | IV (IVa, IVb) | 4 | (4.5) | 4 | (4.7) |
| MG-ADL mean (SD) | | 10.9 | (3.4) | 10.3 | (2.5) |
| OMG score, mean (SD) | | 19.4 | (4.5) | 18.7 | (3.6) |
| MGC score, mean (SD) | | 21.6 | (7.2) | 20.1 | (6.0) |
| MG-QoL15r score, mean (SD) | | 18.9 | (6.8) | 18.6 | (6.6) |
| Prior thymectomy, n (%) | | 37 | (42.0) | 45 | (52.3) |
| Duration of disease, years, mean (SD) | | 9.0 | (10.4) | 9.3 | (9.5) |
| Treatment refractory*, n (%) | | 44 | (50.0) | 44 | (51.2) |
| MG medication | cholinesterase inhibitor | 73 | (83.0) | 74 | (86.0) |
| at baseline^{†}, | corticosteroids | 51 | (58.0) | 59 | (68.6) |
| n (%) | azathioprine, MMF | 35 | (39.8) | 30 | (34.9) |
| | cyclosporin, | 15 | (17.0) | 12 | (14.0) |

cyclophosphamide,
methotrexate,
tacrolimus, rituximab

mITT population unless otherwise stated. The mITT population includes all randomised subjects who received at least one dose of study drug and have at least one post-dosing MG-ADL score. * A participant is considered ‘MG refractory’ if they have had treatment for at least one year with two or more of the following therapies: prednisone, azathioprine, mycophenolate, cyclosporine, cyclophosphamide, methotrexate, tacrolimus, rituximab, eculizumab, or other corticosteroids; OR history of treatment with at least one of the above therapies for 1 year or more and required chronic PLEX, IVIg or SCIg at least every 3 months for the 12 months prior to enrolment. T Safety set. Includes all subjects who received at least one dose of study drug with subjects analysed based on the actual study treatment received.

### Primary Outcome Measures:

Change from Baseline to Week 12 (CFB) in the MG-ADL Score [Time Frame: From Baseline (Day 1) to Week 12]. The MG-ADL profile provides an assessment of MG symptom severity and measures 8 items on a 0-3 scale, with 0 being the least severe. The total sum of the 8 items represents the MG-ADL score. The MG-ADL score can range from 0 (least severe) to 24 (most severe).

### Secondary Outcome Measures:

Change from Baseline to Week 12 in the Quantitative Myasthenia Gravis (QMG) Score [Time Frame: From Baseline (Day 1) to Week 12]. The QMG test is a standardized quantitative strength scoring system and measures 13 items on a 0-3 scale, with 0 being the least severe. The total sum of the 13 items represents the QMG score. The QMG score can range from 0 (least severe) to 39 (most severe).

Change from Baseline to Week 12 in the Myasthenia Gravis Composite (MGC) Score [Time Frame: From Baseline (Day 1) to Week 12]. The MGC is a 10-item scale that has been used to measure the clinical status of patients with Myasthenia Gravis (MG) in order to evaluate treatment response. The MGC has 4-point Likerttype Scale response options ranging from 0 to 2, 3, 4, 5, 6 or 9 according to the item (weighted response options). The total score is the sum of all items (range 0-50) where higher scores indicate more severe impairment due to the disease.

Change from Baseline to Week 12 in the Myasthenia Gravis—Quality of Life revised (MG-QOL15r) Score [Time Frame: From Baseline (Day 1) to Week 12]. The MG-QOL15r is a 15-item survey that was designed to assess quality of life in patients with MG. The MG-QOL has 3-point Likert Scale response options ranging from 0 to 2. The MG-QOL15r score can range from 0 to 30, where higher scores indicate more severe impact of the disease on aspects of the patient's life.

Time to first receipt of rescue therapy over the 12-week Treatment Period [Time Frame: Treatment Period (from Day 1 to Week 12)]. For patients who require rescue treatment, the time from first investigational medicinal product (IMP) to the first dose of rescue treatment will be calculated.

Percentage of participants achieving Minimal Symptom Expression (MSE) at Week 12 [Time Frame: Week 12]. MSE is defined as an MG-ADL of 0 or 1 at Week 12 without rescue therapy. The MG-ADL profile provides an assessment of MG symptom severity and measures 8 items on a 0-3 scale, with 0 being the least severe. The total sum of the 13 items represents the MG-ADL score. The MG-ADL score can range from 0 (least severe) to 24 (most severe).

Percentage of participants achieving a ≥3-point reduction in MG-ADL Score at Week 12 without rescue therapy [Time Frame: Week 12]. The MG-ADL profile provides an assessment of gMG symptom severity and measures 8 items on a 0-3 scale, with 0 being the least severe. The total sum of the 13 items represents the MG-ADL score. The MG-ADL score can range from 0 (least severe) to 24 (most severe). A 3-point change in this assessment is considered clinically meaningful.

Percentage of participants achieving a ≥5-point reduction in QMG Score without rescue therapy at Week 12 [Time Frame: Week 12]. The QMG test is a standardized quantitative strength scoring system and measures 13 items on a 0-3 scale, with 0 being the least severe. The total sum of the 13 items represents the QMG score. The QMG score can range from 0 (least severe) to 39 (most severe). A change in the QMG Score of 3 points or more may be considered clinically meaningful, in a typical clinical trial population of gMG patients.

Incidence of treatment-emergent adverse events (TEAEs) [Time Frame: From Baseline (Day 1) to Safety Follow-Up Visit (up to Week 19)]. A treatment-emergent adverse event is any untoward medical occurrence in a patient or clinical study participant, temporally associated with the use of study medication, whether or not considered related to the study medication.

Of the 174 study participants dosed in this study, 166 completed the study. Overall, zilucoplan showed a favorable safety profile and good tolerability.

### Efficacy of Zilucoplan in Patient Population

This set of data demonstrates efficacy of zilucoplan in a broader patient population, including nonrefractory and/or refractory gMG patients.

The following clinical assessments were performed to evaluate efficacy in the broad patient population: The MG-ADL score was the primary outcome measure, the key secondary outcome measures were the QMG scale, the MGC, and the MG-QOL15r, and the secondary outcome measures were: time to first administration of rescue therapy over the 12-week Treatment Period, achieving minimal symptom expression (MSE), defined as an MG-ADL of 0 or 1 at Week 12 without rescue therapy, achieving a ≥3-point reduction in MG-ADL Score at Week 12 without rescue therapy, and achieving a ≥5-point reduction in QMG Score without rescue therapy at Week 12. The exploratory outcome measures included responder analysis for changes in MG-ADL and QMG composite scores from baseline without rescue therapy. The results are presented below. There was a clinically meaningful and statistically significant improvement in the zilucoplan treatment groups compared to placebo in the primary and key secondary variables. The efficacy results in all other efficacy assessments were in line with the above results showing robust and consistent efficacy of zilucoplan in different aspects of gMG.

Efficacy of zilucoplan across all study participants with generalized myasthenia gravis: MG-ADL score

A clinically meaningful improvement (defined as ≥2 point reduction) and statistically significant improvement from baseline in MG-ADL score was observed at week 12 for the zilucoplan (ZLP) treatment group versus placebo (Table 9). The jump to reference sensitivity analysis (as requested by the European Medicines Agency) was in line with the primary analysis.

TABLE 9
MG-ADL score least squares (LS) mean change from
baseline to week 12 (mITT population) (MG0010).

| | | |
|---|---|---|
| | ZLP | |
| Placebo | 0.3 mg/kg | |
| (N = 88) | (N = 86) | p-value |

Primary analysis^{a}

| | | | |
|---|---|---|---|
| LS mean CFB (95% CI) | −2.30 | −4.42 | <0.001 |
| | (−3.17, −1.43) | (−5.31, −3.53) | |

| | |
|---|---|
| LS mean difference vs | −2.12 |
| placebo (95% CI) | (−3.26, −0.97) |

J2R analysis^{b}

| | | | |
|---|---|---|---|
| LS mean CFB (95% CI) | −2.43 | −4.48 | <0.001 |
| | (−3.30, −1.57) | (−5.36, −3.60) | |

| | |
|---|---|
| LS mean difference vs | −2.05 |
| placebo (95% CI) | (−3.18 −0.91) |

CFB = change from baseline; CI = confidence interval; J2R = jump to reference; LS = least squares; MG = myasthenia gravis; MG-ADL = MG-Activities of Daily Living mITT = modified intent-to-treat; ZLP = zilucoplan.
^{a}Rescue therapy, death or myasthenic crisis were assumed as treatment failure and any data after the intercurrent event was imputed Baseline or last value, whichever is worse. Any other intercurrent event was assumed to be missing at random.
^{b}J2R imputes missing data assuming participants jump to behave like those in the specified reference group (i.e. placebo control arm) following their last observed time point.

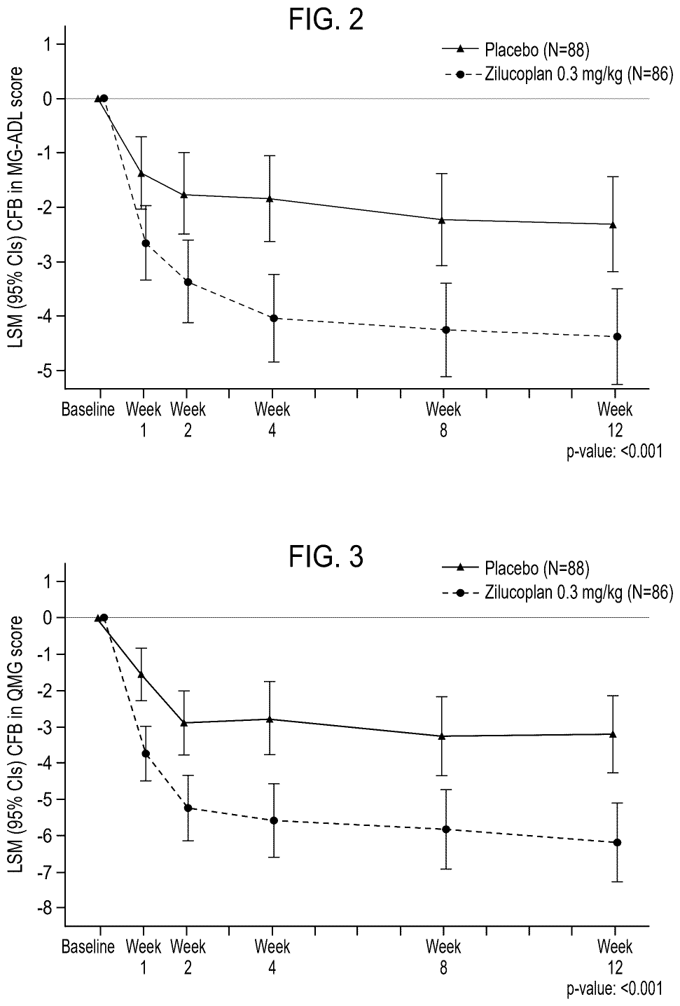

The change from Baseline over time in MG-ADL score is presented for the modified intent to treat (mITT) population in FIG. 2. Rapid separation of the curves started at week 1 and increased through week 4 with stabilization thereafter. This effect was maintained up to week 12 (nominal p<0.05 for LS mean difference for all visits after Baseline). FIG. 2 illustrates that zilucoplan highly statistically significantly and clinically meaningfully reduced MG-ADL from baseline to Week 12, with least squares (LS) mean difference (95% CI) of −2.12 (−3.26, −0.97) vs placebo (p<0.001).

As detailed below, least squares (LS) mean differences for zilucoplan vs placebo at Week 12 for key secondary efficacy endpoints were also highly statistically significant and clinically meaningful: −3.07 (−4.48, −1.66; p<0.001) for QMG, −3.20 (−5.54, −1.16; p=0.0023) for MGC and −2.51 (−4.46, −0.55; p<0.02) for MG-QoL15r. Rapid separation of zilucoplan and placebo curves for the key primary and secondary efficacy endpoints started at Week 1 and increased through Week 4, with stabilization thereafter up to Week 12 (nominal p<0.05 for LS mean difference for all visits after baseline; FIGS. 2-5).

Efficacy of Zilucoplan Across all Study Participants with Generalized Myasthenia Gravis: QMG Score

A clinically meaningful improvement (defined as ≥3 point reduction) and statistically significant improvement from baseline in QMG score was observed at week 12 for the ZLP treatment group versus placebo (Table 10).

TABLE 10
QMG score LS mean change from Baseline
to Week 12 (mITT population)

| | | |
|---|---|---|
| | ZLP | |
| Placebo | 0.3 mg/kg | |
| (N = 88) | (N = 86) | p-value |

Primary analysis^{a}

| | | | |
|---|---|---|---|
| LS mean CFB (95% CI) | −3.23 | −6.30 | <0.001 |
| | (−4.29, −2.18) | (−7.38, −5.22) | |

| | |
|---|---|
| LS mean difference vs | −3.07 |
| placebo (95% CI) | (−4.48, −1.66) |

CFB = change from baseline; CI = confidence interval; LS = least squares; mITT = modified intent-to-treat; QMG = quantitative myasthenia gravis; ZLP = zilucoplan.
^{a}Rescue therapy, death or myasthenic crisis were assumed as treatment failure and any data after the intercurrent event was imputed Baseline or last value, whichever is worse. Any other intercurrent event was assumed to be missing at random.

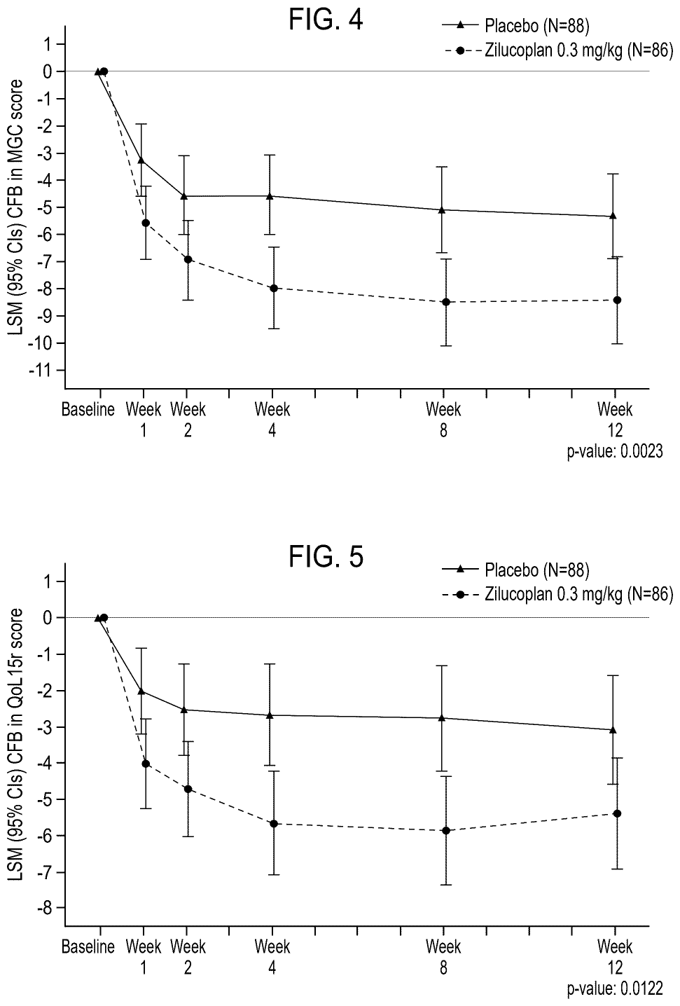

The change from Baseline over time in QMG scores is presented for the mITT Population in (FIG. 3). Rapid separation of the curves started at week 1 and increased through week 4 with stabilisation thereafter. This effect was maintained up to week 12 (nominal p<0.05 for LS mean difference for all visits after Baseline).

Efficacy of Zilucoplan Across all Study Participants with Generalized Myasthenia Gravis: MGC Score

A clinically meaningful improvement (defined as ≥3 point reduction) and statistically significant improvement from baseline in MGC score was observed at week 12 for the ZLP treatment group versus placebo (Table 11)

TABLE 11
MGC score LS mean change from baseline
to week 12 (mITT population).

| | | |
|---|---|---|
| | ZLP | |
| Placebo | 0.3 mg/kg | |
| (N = 88) | (N = 86) | p-value |

Primary analysis^{a}

| | | | |
|---|---|---|---|
| LS mean CFB (95% CI) | −5.42 | −8.62 | 0.0023 |
| | (−6.98, −3.86) | (−10.22, −7.01) | |

| | |
|---|---|
| LS mean difference vs | −3.20 |
| placebo (95% CI) | (−5.24, −1.61) |

CFB = change from baseline; CI = confidence interval; LS = least squares; MGC = myasthenia gravis composite; mITT = modified intent-to-treat; ZLP = zilucoplan.
^{a}Rescue therapy, death or myasthenic crisis were assumed as treatment failure and any data after the intercurrent event was imputed Baseline or last value, whichever is worse.

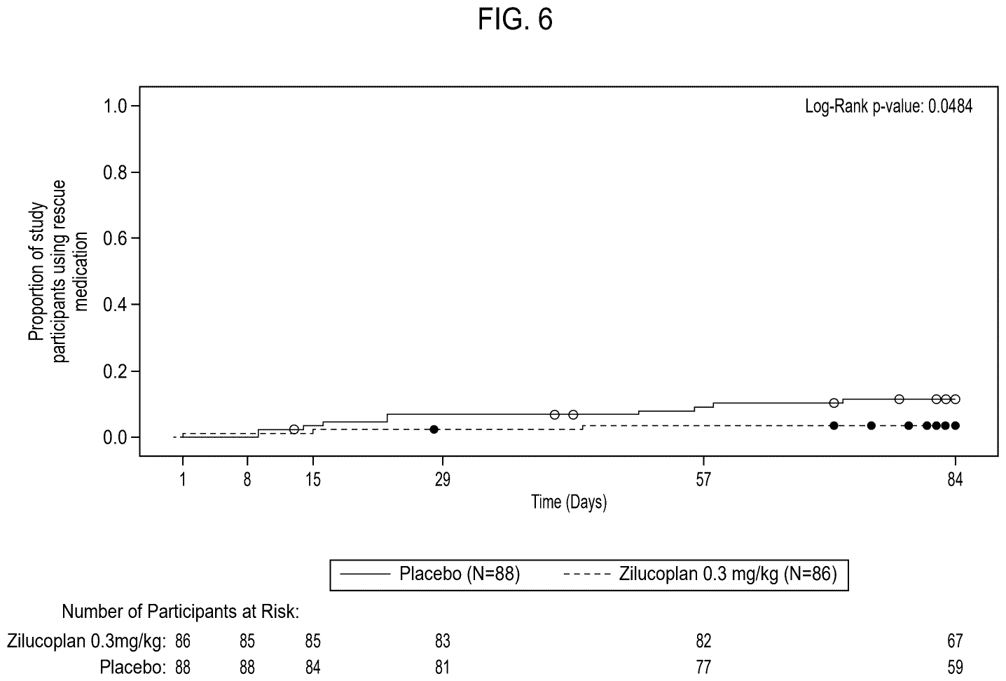

The change from Baseline over time in MGC scores is presented for the mITT population in (FIG. 4). Rapid separation of the curves started at week 1 and increased through week 4 with stabilization thereafter. This effect was maintained up to week 12 (nominal p<0.05 for LS mean difference for all visits after baseline).

Efficacy of Zilucoplan Across all Study Participants with Generalized Myasthenia Gravis: MG QOL15r Score.

Statistically significant improvement from baseline in MG QOL15r score was observed at week 12 for the ZLP treatment group versus placebo (Table 12). A threshold for minimal clinically important difference (MCID) has not yet been defined for MG QOL15r.

TABLE 12
MG-QOL15r score LS mean change from Baseline
to Week 12 (mITT population).

| | | |
|---|---|---|
| | ZLP | |
| Placebo | 0.3 mg/kg | |
| (N = 88) | (N = 86) | p-value |

Primary analysis^{a}

| | | | |
|---|---|---|---|
| LS mean CFB (95% CI) | −3.16 | −5.66 | 0.0122 |
| | (−4.65, −1.66) | (−7.19, −4.14) | |

| | |
|---|---|
| LS mean difference vs | −2.51 |
| placebo (95% CI) | (−4.46, −0.55) |

CFB = change from baseline; CI = confidence interval; LS = least squares; MG-QOL15r = myasthenia gravis quality of life 15 revised; mITT = modified intent-to-treat; ZLP = zilucoplan.
^{a}Rescue therapy, death or myasthenic crisis were assumed as treatment failure and any data after the intercurrent event was imputed Baseline or last value, whichever is worse.

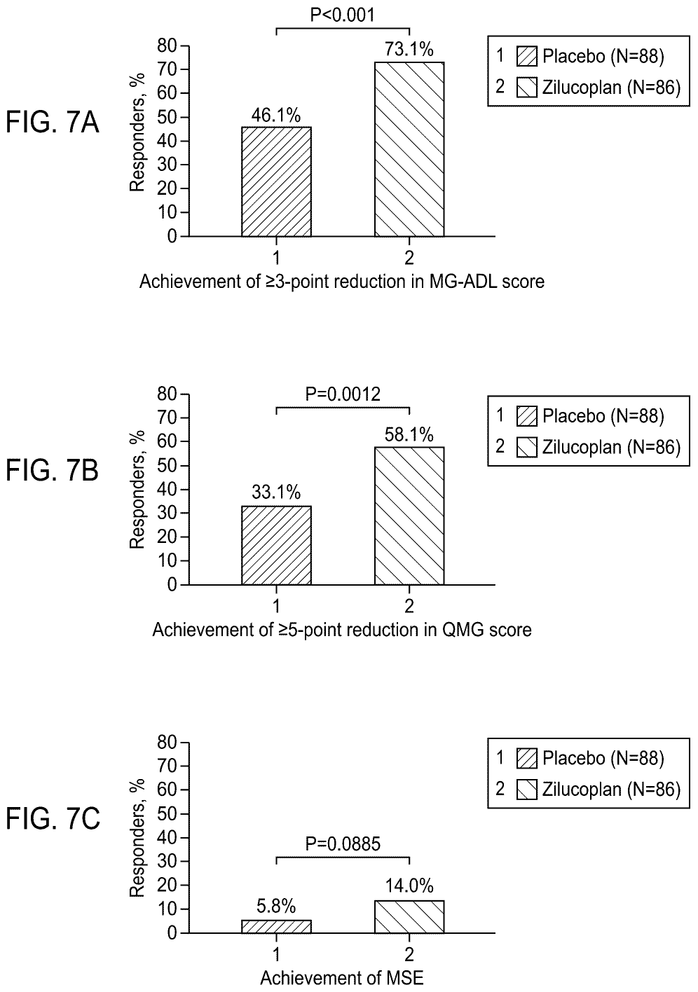

The change from Baseline over time in MG-QOL15r scores is presented for the mITT population in (FIG. 5). Rapid separation of the curves started at week 1 and increased through week 4 with stabilization thereafter. This effect was maintained up to week 12 (nominal p<0.05 for LS mean difference for all visits after baseline). Of note, no threshold or minimally clinically important difference has been established for this scale.

### Secondary Endpoint: Time to First Receipt of Rescue Therapy

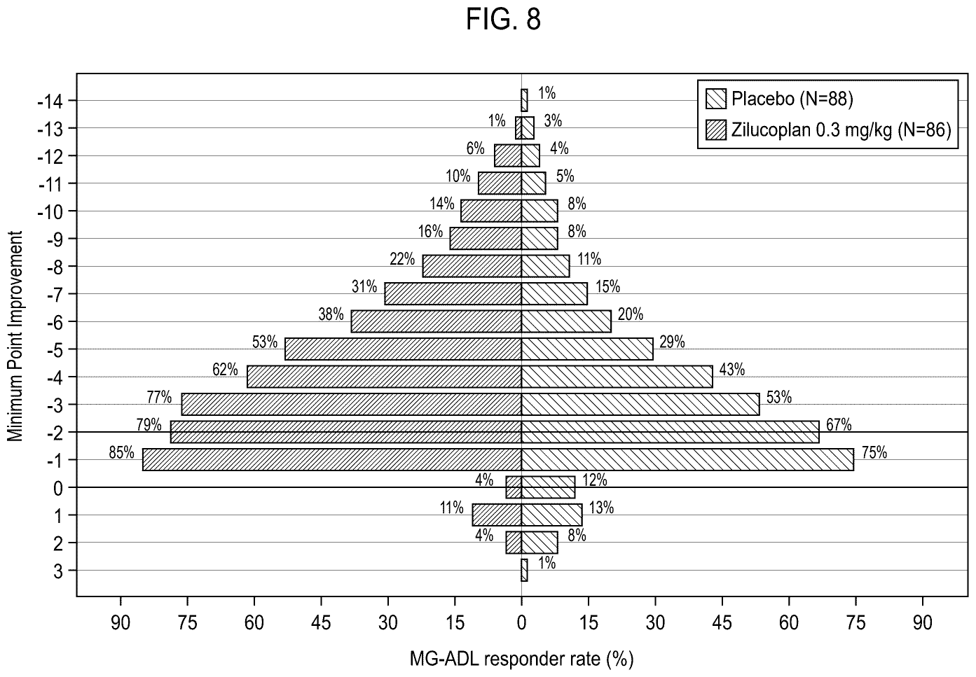

There were substantially fewer participants receiving rescue therapy by Week 12 in the ZLP treatment group compared with placebo (Table 13 and FIG. 6).

TABLE 13
Time to first receipt of rescue therapy (mITT population)

| | | ZLP |
|---|---|---|
| | Placebo | 0.3 mg/kg |
| | (N = 88) | (N = 86) |
| Cumulative number of events at Week 12 | 10 | 3 |
| Cumulative proportion of participants | 11.4 | 3.5 |
| using rescue therapy at Week 12 (%) | | |

mITT = modified intent-to-treat; ZLP = zilucoplan.

### Secondary Endpoint: Responder Rates

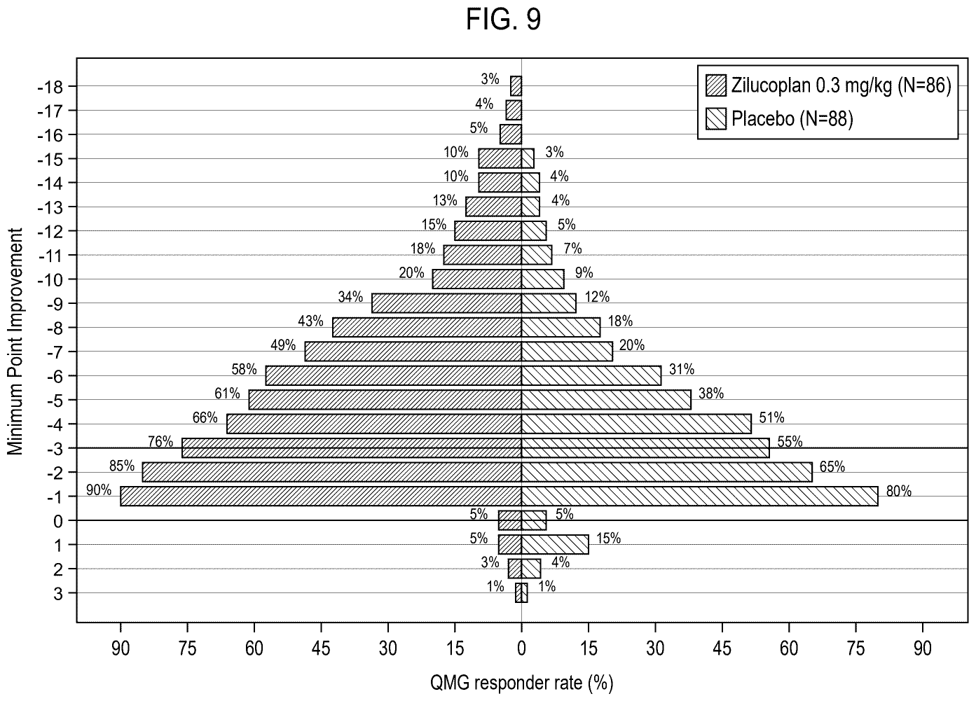

FIGS. 7A-7C illustrates responder rates for MG-ADL (FIG. 7A), QMG score (FIG. 7B), and minimal symptom expression (MSE) (FIG. 7C) without rescue therapy at week 12 for placebo versus zilucoplan (ZLP). Significantly more participants treated with ZLP achieved a ≥3 point reduction in MG-ADL score (73.1% versus 46.1%, p<0.001) (FIG. 7A) and a ≥5 point reduction in QMG score (58.1% versus 33.1%, p=0.0012) (FIG. 7B) at week 12 compared with placebo. Substantially more participants in the ZLP arm achieved MSE (MG-ADL score of 0 or 1) compared with placebo (14.0% versus 5.8%, respectively) (FIG. 7C). Compared to eculizumab at week 26, numerically higher rates at week 12 with ZLP are observed (Phase 3 Study REGAIN, Howard et al., 2017).

### Exploratory Endpoint: Responder Analysis for Changes in MG-ADL

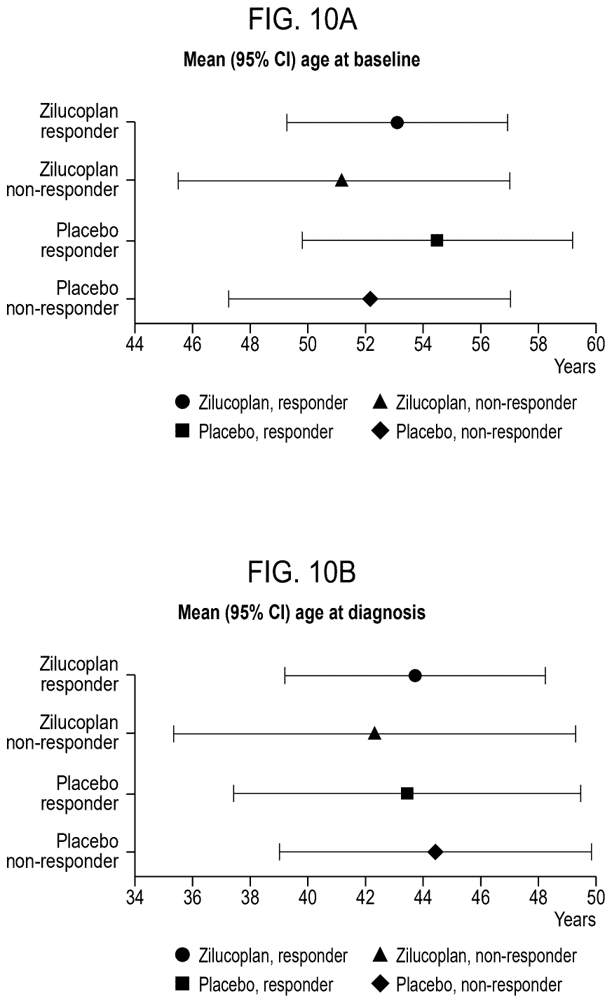

FIG. 8 shows a responder analysis for changes in MG-ADL score at week 12 in the modified intent to treat (mITT) population of the RAISE clinical study. The graph of FIG. 8 shows minimum point improvements in MG-ADL score, i.e., a scale of negative values representing larger score decreases from baseline. A greater proportion of participants receiving ZLP showed improvement in MG-ADL score response at week 12 compared with placebo (FIG. 8). Participants who received rescue medication are classified as non-responders after the first rescue medication administration.

### Exploratory Endpoint: Responder Analysis for Changes in QMG

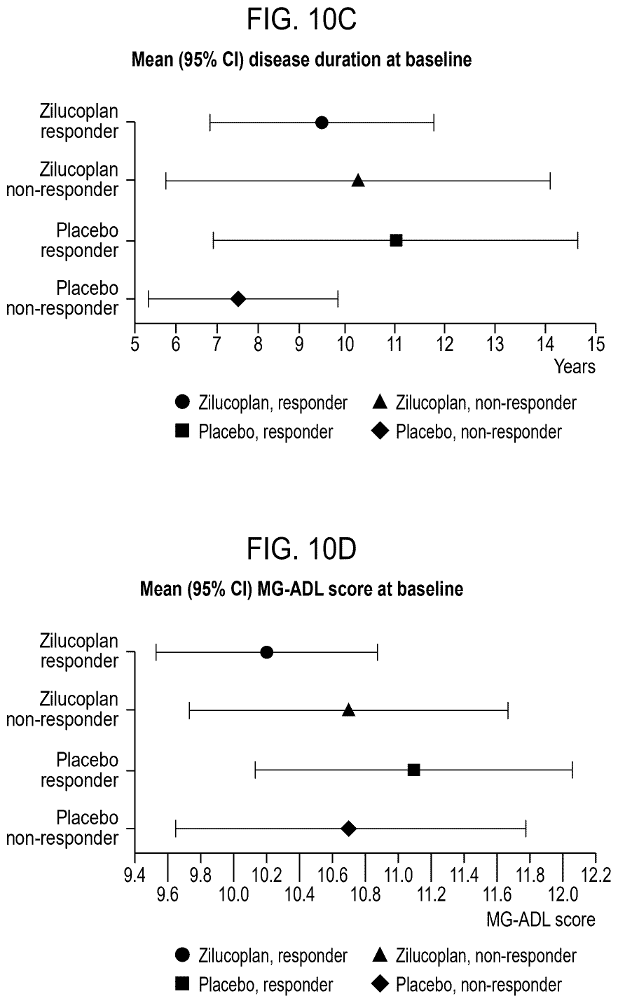

FIG. 9 shows a responder analysis for changes in QMG score at week 12 in the modified intent to treat (mITT) population of the RAISE clinical study. Participants who received rescue medication are classified as non-responders after the first rescue medication administration. A greater proportion of participants receiving ZLP showed improvement in QMG response at week 12 compared with placebo (FIG. 9).

MG-ADL and QMG response rates for each level of improvement are shown in FIGS. 8-9. Logistic regression analyses show 76.5% of zilucoplan patients achieved a 3-point improvement in MG-ADL vs 46.1% in the placebo arm (OR [95% CI]=3.182 [1.660, 6.098]; p<0.001). A ≥5-point improvement in QMG was seen in 58.0% of patients receiving zilucoplan vs 33.1% in the placebo arm (OR [95% CI]=2.869 [1.520, 5.418]; p=0.0012).

MG-ADL results in subgroups based on demographics or disease characteristics at baseline mirrored those seen in the overall population. CFB in MG-ADL at Week 12 with zilucoplan vs placebo for: age (<65 years: −4.56 vs −2.75; ≥65 years: −5.14 vs −3.08), gender (male: −5.12 vs −2.85; female: −4.43 vs −2.84), baseline MG-ADL (≤9: −3.88 vs −2.48; ≥10: −5.24 vs −3.06) and region (North America: −4.67 vs −3.61; Europe: −4.74 vs −2.31; Asia: −4.71 vs −1.00).

### Further Analysis

Adults with AChR positive gMG (MGFA Disease Class II-IV) were randomized 1:1 to daily subcutaneous zilucoplan (ZLP) 0.3 mg/kg (n=86) or placebo (PBO; n=88) for 12 weeks. MG-ADL and QMG responders (≥3- and ≥5-point improvement, respectively) were assessed and a post hoc analysis of baseline disease characteristics on treatment response was performed. Week 12 responder rates were higher for ZLP vs PBO (MG-ADL: 73% vs 46%, p<0.001; QMG: 58% vs 33%, p=0.0012). ZLP showed rapid onset of efficacy: at Week 1, 45% and 32% of ZLP patients were MG-ADL and QMG responders (PBO: 30% and 8%). There were no meaningful differences in baseline disease characteristics of MG-ADL responders and non-responders at Week 12, MG-ADL responder rate continued to increase up to Week 12.

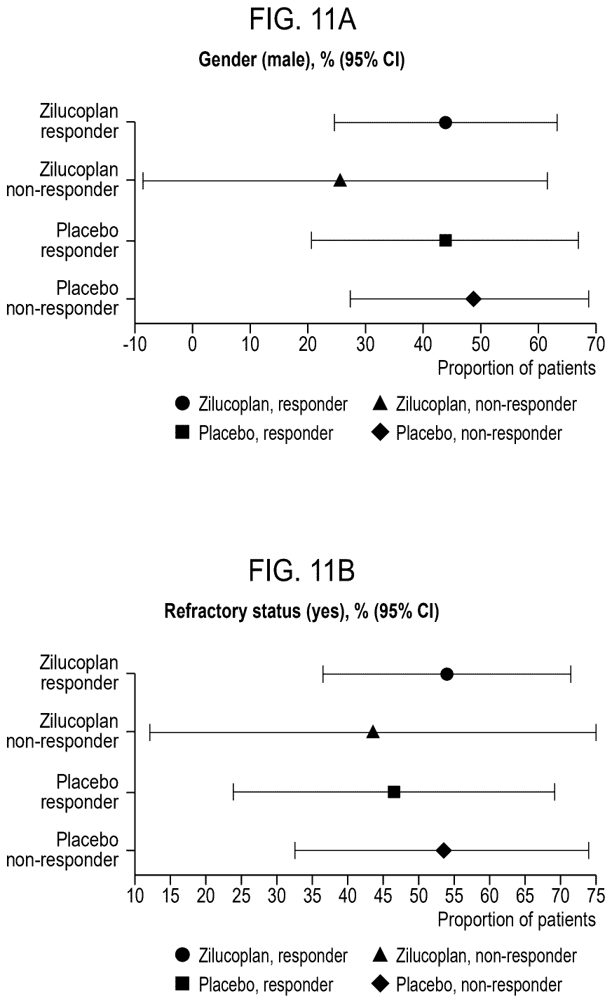

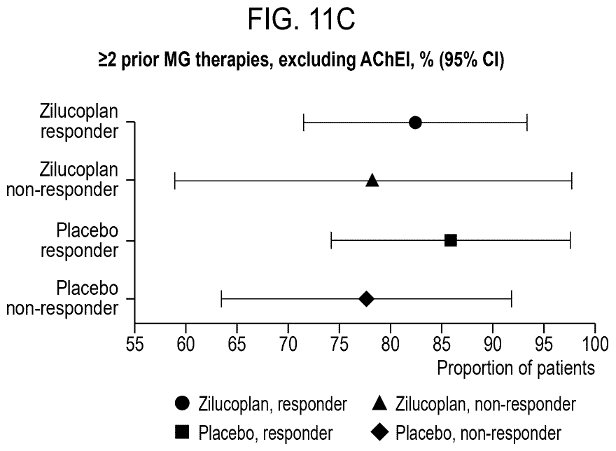

FIGS. 10A-10D and FIGS. 11A-11C illustrate baseline characteristics for MG-ADL responders and non-responders at Week 12 (zilucoplan responders n=63, zilucoplan non-responders n=23, placebo responders n=43, placebo non-responders n=45). FIGS. 10A-10D, continuous characteristics. FIG. 10A, age as baseline. FIG. 10B, age at diagnosis. FIG. 10C, disease duration at baseline. FIG. 10D, MG-ADL score at baseline. CI, confidence interval. FIGS. 11A-11C, categorical characteristics. FIG. 11A, gender (male). FIG. 11B, refractory status (yes). FIG. 11C, at least 2 prior MG therapies, excluding acetylcholinesterase inhibitor (AChEI). CIs are not adjusted for covariates or multiple testing. CI, confidence interval.

### Extension Study

MG0011 is a multicenter, open label extension study to evaluate the long-term efficacy, safety, and tolerability of ZLP in study participants with gMG who have previously participated in a qualifying ZLP study. Study participants receive ZLP 0.3 mg/kg administered SC until ZLP is approved and available in the territory, or UCB terminates development of ZLP for gMG. All study participants who completed the study MG0010 decided to enter the study MG0011. The good rollover rate is indicative of patients and clinicians' preference to continue long-term treatment.

### SUMMARY

The approval of eculizumab was a significant advance, but does not address the needs of all gMG patients. Many patients still experience significant disease burden (QMG≥12 or MG-ADL≥6), regardless of current treatment.

Retrospective analysis of the MG-ADL and QMG scores recorded during the eculizumab REGAIN study and its open-label extension (OLE) has shown that there is still a non-negligible portion of participants who do not respond to the drug and/or are long responders (Howard et. al., 2017) and thus for whom eculizumab may not be the best option, showing the need for alternative therapies. For instance, between Week 12 and the end of the OLE of eculizumab treatment, an MG-ADL response had been achieved by 17% of participants (MG-ADL late responders) and 15% of participants did not achieve the defined MG-ADL response during eculizumab treatment (MG-ADL non responders). With regard to QMG, 15% of participants had achieved a QMG response between Week 12 and the end of the OLE (QMG late responders); and 29% of participants did not achieve the defined QMG response during eculizumab treatment (QMG non responders).

In the double blind portion of the REGAIN study, about 40 to 55% of randomized participants were classified as non-responders at Week 26 (<3 points reduction in MG-ADL or <5 points reduction in QMG [Howard, 2017]).

The zilucoplan Phase 3 study described above studied a broad population of moderate to severe participants with gMG regardless of prior treatment history.

Zilucoplan showed consistent, statistically significant and clinically meaningful benefits across all clinical endpoints (MG-ADL, QMG, MG-QOL15r and MGC), and a favorable safety and tolerability profile in a broad range of study participants living with gMG. This reinforces the role of the terminal complement cascade in the pathogenesis of gMG in all AChR positive participants, not just those deemed ‘refractory’ to SOC therapies.

The primary endpoint of the trial was met; a clinically meaningful (MCID=2) and statistically significant improvement from baseline in MG-ADL score at week 12 was observed for the zilucoplan treatment group vs placebo. All key secondary endpoints of the trial were met; statistically significant improvements from baseline in QMG, MGC and MG-QoL15r scores were observed at week 12 for the zilucoplan treatment group vs placebo.

Overall, zilucoplan showed a favorable safety profile and good tolerability; there were no major safety findings. The incidence of serious TEAEs in the zilucoplan and placebo treatment arms was similar.

The data and analysis presented herein clearly demonstrates efficacy of zilucoplan in a broad patient population, including nonrefractory and/or refractory gMG patients.

### Efficacy of Zilucoplan in Non-Refractory gMG Versus Refractory gMG Patients

In this Phase 3 gMG randomized study, ZLP 0.3 mg/kg dose demonstrated a significant benefit in a broad gMG study participant population, which included approximately 50% nonrefractory study participants (compared to the refractory population as defined herein). As per the inclusion criteria for the eculizumab Phase 3 study (REGAIN study), treatment refractory status is defined as: (clintrials.gov)
a. Failed treatment over 1 year or more with 2 or more immunosuppressive therapies (ISTs) either in combination or as monotherapy (i.e., continued to have impairment of activities of daily living [persistent weakness, experienced crisis, or unable to tolerate IST] despite ISTs); or
b. Failed at least 1 IST and required chronic plasmapheresis, plasma exchange, or IVIG to control symptoms (i.e., study participants who required plasma exchange or IVIG on a regular basis for the management of muscle weakness at least every 3 months over the previous 12 months).

Immunosuppressive therapies included, but were not limited to, corticosteroids, azathioprine, mycophenolate mofetil, methotrexate, cyclosporine, tacrolimus, or cyclophosphamide.

The ZLP Phase 3 study defines its refractory and non-refractory populations in accordance with the same entry criteria.

In order to understand whether treatment response differs significantly between those who would be considered ‘refractory’ (as per the definition in the eculizumab program) and those who would be considered non-refractory, a prespecified subgroup analysis was performed. This analysis showed variability in the observed effect size in some endpoints, however the overall data demonstrated the efficacy of ZLP in both subpopulations (Table 14). In particular for QMG, considered to be a more objective physician-reported measure compared to MG-ADL, which is a patient-reported outcome.

TABLE 14
Change from baseline in clinical efficacy outcomes in refractory
and non-refractory populations in MG0010 (mITT population).

| | | | | ZLP |
|---|---|---|---|---|
| | Refractory | | Placebo | 0.3 mg/kg |
| Parameter | Status | Statistic | (N = 88) | (N = 86) |
| MG-ADL | Yes | n | 44 | 44 |
| | | LS mean CFB (SE) | −1.62 (0.58) | −4.73 (0.58) |
| | | LS mean (SE) | −3.11 (0.80) | |
| | | difference vs | (−4.69, −1.52) | |
| | | placebo (95% CI) | | |
| | No | n | 44 | 42 |
| | | LS mean CFB (SE) | −2.19 (0.87) | −3.36 (0.88) |
| | | LS mean (SE) | −1.17 (0.83) | |
| | | difference vs | (−2.83, 0.48) | |
| | | placebo (95% CI) | | |
| QMG | Yes | n | 44 | 40 |
| | | LS mean CFB (SE) | −2.76 (0.75) | −6.08 (0.76) |
| | | LS mean (SE) | −3.32 (1.05) | |
| | | difference vs | (−5.42, −1.23) | |
| | | placebo (95% CI) | | |
| | No | n | 44 | 40 |
| | | LS mean CFB (SE) | −3.48 (0.96) | −6.35 (0.97) |
| | | LS mean (SE) | −2.86 (0.97) | |
| | | difference vs | (−4.79, −0.94) | |
| | | placebo (95% CI) | | |
| MGC | Yes | n | 44 | 40 |
| | | LS mean CFB (SE) | −4.25 (1.08) | −8.02 (1.09) |
| | | LS mean (SE) | −3.76 (1.50) | |
| | | difference vs | (−6.75, −0.78) | |
| | | placebo (95% CI) | | |
| | No | n | 44 | 40 |
| | | LS mean CFB (SE) | −5.58 (1.58) | −8.33 (1.61) |
| | | LS mean (SE) | −2.76 (1.41) | |
| | | difference vs | (−5.56, 0.05) | |
| | | placebo (95% CI) | | |
| MG- | Yes | n | 44 | 40 |
| QOL15r | | LS mean CFB (SE) | −2.35 (0.94) | −5.70 (0.95) |
| | | LS mean (SE) | −3.35 (1.30) | |
| | | difference vs | (−5.94, −0.76) | |
| | | placebo (95% CI) | | |
| | No | n | 44 | 40 |
| | | LS mean CFB (SE) | −3.00 (1.57) | −4.68 (1.59) |

| | |
|---|---|
| LS mean (SE) | −1.68 (1.48) |
| difference vs | (−4.62, 1.25) |
| placebo (95% CI) | |

CFB = change from baseline; CI = confidence interval; LS = least squares.

TABLE 15
Summary of MG-ADL and QMG responder rates in refractory and
non-refractory populations in MG0010 (mITT population).

| | | | ZLP | |
|---|---|---|---|---|
| | | Placebo | 0.3 mg/kg | |
| | Refractory | (N = 88) | (N = 86) | |
| Responder | Status | n/NSub (%) | n/NSub (%) | |
| ≥3 point reduction | Yes | 17/42 (40.5%) | 33/44 | (75%) |
| in MG-ADL score | No | 23/43 (53.5%) | 29/40 | (72.5%) |
| at Week 12 | | | | |
| ≥5 point reduction | Yes | 11/41 (26.8%) | 24/43 | (55.8%) |
| in QMG score at | No | 17/43 (39.5%) | 25/40 | (62.5%) |
| Week 12 | | | | |

mITT = mITT = modified intent to treat; n = number of responders. NSub = number of subjects with available information at the timepoint. Percentages are based on Nsub.

These data suggest that zilucoplan can provide an additional clinical benefit to a broad population of patients

### Improved Efficacy of Zilucoplan Versus Eculizumab in Patients Having Refractory gMG

Analysis of the clinical results data of this study shows the efficacy of zilucoplan in treating refractory gMG patients. In addition, these clinical results were compared to the results obtained using eculizumab in the REGAIN study of refractory gMG treatment.

Although cross-study comparisons should be performed with caution, it is noteworthy that the clinical effect of zilucoplan administered at 0.3 mg/kg daily SC as measured by the MG-ADL and QMG at Week 12 in the refractory population is numerically higher as compared to the effect observed with eculizumab at Week 26 in the REGAIN study (Table 16).

TABLE 16
Comparison of main clinical efficacy outcomes between zilucoplan
in Phase 3 Study MG0010 at Week 12 (refractory population)
and eculizumab in Phase 3 Study REGAIN at Week 26.

| | LS mean difference vs placebo (CI); p-value ^{a} | |
|---|---|---|
| | ZLP 0.3 mg/kg | Eculizumab 1200 mg |
| MG-ADL | −3.11 (−4.69, −1.52); <0.001 | −1.8 (−3.2, −0.5); 0.0077 |
| QMG | −3.32 (−5.42, −1.23); 0.0022 | −2.9 (−4.6, −1.2); 0.0007 |
| MGC | −3.76 (−6.75, −0.78); 0.0142 | −3.3 (−5.9, −0.6); 0.0168 |

Note:
Data for MG-QOL is not represented given different scales were applied (MG-QOL15 vs MG-QOL15r).
^{a} pre-specified analysis with nominal p-values for ZLP 0.3 mg/kg.
Source: Phase 3 Study REGAIN (Howard et al, 2017)

These results indicate that even for patients having refractory gMG, a consistent and greater clinical benefit is achieved in a shorter period of time using zilucoplan over eculizumab.

### EQUIVALENTS AND INCORPORATION BY REFERENCE

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

1. A method of treating refractory generalized myasthenia gravis (gMG) in a human patient in need thereof, the method comprising:
administering a therapeutically effective amount of zilucoplan to a human patient identified as (i) positive for auto-antibodies binding to nicotinic acetylcholine receptor (anti-AChR) and (ii) having refractory gMG.

2. The method of claim 1, wherein, prior to administration, the patient is refractory to treatment for 1 year or more with immunosuppressant therapy (IST) and requires chronic plasma exchange or chronic IVIG to maintain clinical stability.

3. The method of claim 1, wherein the patient experiences a reduction in Myasthenia Gravis Activities of Daily Living (MG-ADL) score of at least 3 points from baseline after 12 weeks of treatment.

4. The method of claim 1, wherein the patient experiences reduction in MG-ADL score of at least 2 points from baseline after 8 weeks of treatment.

5. The method of claim 1, wherein the patient experiences a clinically meaningful improvement (reduction) in quantitative Myasthenia Gravis (QMG) score, in Myasthenia Gravis Composite (MGC) score, or in quality of life as measured by Myasthenia Gravis Quality of Life revised (MG-QOL-15r) score, after 12 weeks of treatment.

6. The method of claim 5, wherein the patient experiences a reduction in QMG score of at least 3 points from baseline after 12 weeks of treatment.

7. The method of claim 6, wherein patient experiences a reduction in QMG score of at least 3 points from baseline after 8 weeks of treatment.

8. The method of claim 5, wherein the patient experiences a reduction in MGC score of at least 3 points from baseline after 12 weeks of treatment.

9. The method of claim 5, wherein the patient experiences a reduction in MG-QOL-15r score of at least 2-points from baseline after 12 weeks of treatment.

10. The method of claim 1, wherein zilucoplan is subcutaneously administered daily to the patient for 12 weeks or more.

11. The method of claim 10, wherein zilucoplan is administered at a daily dose of from about 0.1 mg/kg (mg zilucoplan/kg subject body weight) to about 0.6 mg/kg.

12. The method of claim 11, wherein zilucoplan is administered at a daily dose of 0.3 mg/kg.

13. The method of claim 1, wherein zilucoplan is administered using a self-administration device comprising a prefilled syringe comprising a preservative-free 40 mg/mL aqueous solution of zilucoplan, or a sodium salt form thereof, having a volume of from about 0.15 ml to about 0.81 mL.

14. The method of claim 1, wherein the patient is between 18 and 85 years old.

15. The method of claim 1, wherein the patient does not need or receive rescue therapy during zilucoplan administration.

16. The method of claim 1, wherein zilucoplan administration is carried out at an MG disease stage that is prior to a critical or crisis stage of MG.

17. The method of claim 1, wherein the patient simultaneously receives standard of care gMG therapy over the course of zilucoplan treatment, wherein the standard of care gMG therapy comprises one or more of cholinesterase inhibitor treatment, pyridostigmine treatment, corticosteroid treatment, and IST.

18. The method of claim 1, further comprising co-administering to the patient a therapeutically effective amount of an additional therapeutic agent.

19. The method of claim 18, wherein the additional therapeutic agent is selected from:
an immunosuppressive agent selected from azathioprine, cyclosporine, cyclosporine A, mycophenolate mofetil, methotrexate, tacrolimus, cyclophosphamide, and rituximab; and
an inhibitor of autoantibody-mediated tissue destruction comprising a neonatal Fc receptor (FcRN) inhibitor, wherein administration of the FcRN inhibitor comprises intravenous immunoglobulin (IVIG) treatment.


SEQUENCE LISTING (1 sequence)

SEQ ID NO: 1 - protein, 15 aa, synthetic construct
  SITE 1: note = Acetylated residue
  REGION 1..6: note = Lactam bridge between residues
  SITE 7: note = N-methyl-aspartic acid
  SITE 8: note = Tert-butylglycine
  SITE 10: note = 7-azatryptophan
  SITE 14: note = Cyclohexylglycine
  SITE 15: note = N-epsilon-(Polyethylene glycol24-gamma-glutamic acid-N-alpha-hexadecanoyl) lysine
KVERFDDXYXEYPXX